SEQUENCE LISTING (25 sequences)

SEQ ID NO: 1 - DNA, 1071 bp, Vanilla planifolia
atggcagctaagctcctcttcttcctactcttcctggtctccgccctctccgtcgcgctcgccggtttcgaagaagacaatccaatccggtccgttacacaaaggcctgactcgattgagcctgccatcctcggcgtccttggcagttgccgccacgccttccacttcgcacggttcgcccgcaggtacgggaagagctacggatcggaggaggagatcaagaagaggttcgggatcttcgtggagaatctagcgtttatccggtccactaatcggaaggatctgtcgtataccctaggaatcaaccaattcgccgacctgacctgggaggaattccggaccaatcgccttggtgcggcgcagaactgctcggcgactgcgcatggaaaccaccggtttgtcgatggcgtgcttcctgtaacgagggattggagggagcaagggatagtgagccctgtaaaggaccaaggaagctgtggatcttgctggactttcagtactactggagcactagaggctgcatatacacagctaactggaaagagcacatcattatctgaacagcaacttgtggactgtgcctcagcattcaataactttggatgcaatggaggtttgccttcccaagcctttgaatacgttaagtacaatggaggcatcgacacagaacagacttatccataccttggtgtcaatggtatctgcaacttcaagcaggagaatgttggtgtcaaggtcattgattcgataaacatcaccctgggtgctgaggatgagttgaagcatgcagtgggcttggtgcgtccagttagcgttgcatttgaggttgtgaaaggtttcaatctgtacaagaaaggtgtatacagcagtgacacctgtggaagagatccaatggatgtgaaccacgcagttcttgccgtcggttatggagtcgaggacgggattccttattggctcatcaagaactcatggggtacaaattggggtgacaatggctactttaagatggaactcggcaagaacatgtgtggtgttgcaacttgcgcatcttatcccattgtggctgtgtag

SEQ ID NO: 2 - protein, 352 aa, Vanilla planifolia
MAAKLLFFLLFLVSALSVALAGFEEDNPIRSVTQRPDSIEPAILGVLGSCRHAFHFARFARRYGKSYGSEEEIKKRFGIFVENLAFIRSTNRKDLSYTLGINQFADLTWEEFRTNRLGAAQNCSATAHGNHRFVDGVLPVTRDWREQGIVSPVKDQGSCGSWTFSTTGALEAAYTQLTGSTLSEQQLVDCASAFNNFGCGGLPSQAFEYVKYNGGIDTEQTYPYLGVMGICNFKQENVGVKVIDSINITLGAEDELKHAVGLVRPVSVAFEVVKGFNLYKKGVYSSDTCGRDPMDVNHAVLAVGYGVEDGIPYWLIKNSWGTNWGDNGYFKMELGKNMCGVATCASYPIVAV

SEQ ID NO: 3 - protein, 7 aa, Artificial Sequence
  Novel Sequence
GVLPVTR

SEQ ID NO: 4 - protein, 13 aa, Artificial Sequence
  Novel Sequence
NSWGTNWGDNGYF

SEQ ID NO: 5 - protein, 6 aa, Artificial Sequence
  Novel Sequence
GFNLYK

SEQ ID NO: 6 - protein, 8 aa, Artificial Sequence
  Novel Sequence
QGIVSPVK

SEQ ID NO: 7 - DNA, 20 bp, Artificial Sequence
  Primer 1a 5
ggngtnctnccngtnacncg

SEQ ID NO: 8 - DNA, 20 bp, Artificial Sequence
  Primer 1a 5
cgngtnacnggnagnacncc

SEQ ID NO: 9 - DNA, 41 bp, Artificial Sequence
  Primer 2a 5
aantcntggggnacnaantggggnganaanggntanttnaa

SEQ ID NO: 10 - DNA, 42 bp, Artificial Sequence
  Primer 2b 5
nttnaantanccnttntcnccccanttngtnccccangantt

SEQ ID NO: 11 - DNA, 17 bp, Artificial Sequence
  Primer 3a 5
ggnttnaanctntanaa

SEQ ID NO: 12 - DNA, 18 bp, Artificial Sequence
  Primer 3b5
nttntanagnttnaancc

SEQ ID NO: 13 - DNA, 31 bp, Artificial Sequence
  4HBS P1a
ggaattccatatggcagctaagctcctcttc

SEQ ID NO: 14 - DNA, 27 bp, Artificial Sequence
  4HBS P1b
cgcggatccctacacagccacaatggg

SEQ ID NO: 15 - DNA, 29 bp, Artificial Sequence
  4HBS P2a
cccatatgcttcctgtaacgagggattgg

SEQ ID NO: 16 - DNA, 30 bp, Artificial Sequence
  4HBS P3b
cccctcgagatggcagctaagctcctcttc

SEQ ID NO: 17 - DNA, 28 bp, Artificial Sequence
  4HBS P3b
ccccactagtctacacagccacaatggg

SEQ ID NO: 18 - DNA, 34 bp, Artificial Sequence
  Oligo 1
gtatctgagctcaaaaatggcagctaagctcctc

SEQ ID NO: 19 - DNA, 34 bp, Artificial Sequence
  Oligo 2
catagaggatccctacacagccacaatgggataa

SEQ ID NO: 20 - protein, 360 aa, Arabidopsis thaliana
MSAKTILSSVVLVVLVAASAAANIGFDESNPIRMVSDGLREVEESVSQILGQSRHVLSFARFTHRYGKKYQNVEEMKLRFSIFKENLDLIRSTNKKGLSYKLGVNQFADLTWQEFQRTKLGAAQNCSATLKGSHKVTEAALPETKDWREDGIVSPVKDQGGCGSCWTFSTTGALEAAYHQAFAFGKGISLSEQQLVDCAGAFNNYGCNGGLPSQAFEYIKSNGGLDTEKAYPYTGKDETCKFSAENVGVQVLNSVSITLGAEDELKHAVGLVRPVSIAFEVIHSFRLYKSGVYTDSHCGSTPMDVNHAVLAVGYGVEDGVPYWLIKNSWGADWGDKGYFKMEMGKNMCGIATCASYPVVA

SEQ ID NO: 21 - protein, 362 aa, Hordeum vulgare
MAHARVLLLALAVLATAAVAVASSSSFADSNPIRPVTDRAASTLESAVLGALGRTRHALRFARFAVRYGKSYESAAEVRRRFRIFSESLEEVRSTNRKGLPYRLGINRFSDMSWEEFQATRLGAAQTCSATKGNHLMRDAAALPETKDWREDGIVSPVKNQAHCGSCWTFSTTGALEAAYTQATGKNISLSEQQLVDCAGGFNNFGCNGGLPSQAFEYIKYNGGIDTEESYPYKGVNGVCHYKAENAAVQVLDSVNITLNAEDELKNAVGLVRPVSVAAFQVIDGFRQYKSGVYTSDHCGTTPDDVNHAVLAVGYGVENGVPYWLIKNSWGADWGDNGYFKMEMGKNMCAIATCASYPVVAA

SEQ ID NO: 22 - protein, 360 aa, Nicotiana tobaccum
MSRFSLLLALVVAGGLFASALAGPATFADENPIRQVVSDGLHELENAILQVVGKTRHALSFARFAHRYGKRYESVEEIKQRFEVFLDNLKMIRSHNKKGLSYKLGVNEFTDLTWDEFRRDRLGAAQNCSATTKGNLKVTNVVLPETKDWREAGIVSPVKNQGKCGSCWTFSTTGALEAAYSQAFGKGISLSEQQLVDCAGAFNNFGCNGGLPSQAFEYIKSNGGLDTEEAYPYTGKNGLCKFSSENVGVKVIDSVNITLGAEDELKYAVALVRPVSIAFEVIKGFKQYKSGVYTSTECGNTPMDVNHAVLAVGYGVEDGVPYWLIKNSWGADWGDEGYFKMEMGKNMCGVATCASYPVVA

SEQ ID NO: 23 - protein, 360 aa, Zea mays
MAPRRLLVLAVVALAATAAAANSGFADSNPIRPVTDRAASALESTVFAALGRTRDALRFARFAVRYGKSYESAAEVHKRFRIFSESLQLVRSTNRKGLSYRLGINRFADMSWEEFRATRLGAAQNCSATLTGNHRMRAAAVALPETKDWREDGIVSPVKNQGHCGSCWTFSTTGALEAAYTQATGKPISLSEQQLVDCGLAFNNFGCNGGLPSQAFEYIKYNGGLDTEESYPYQGVNGISKFKNENVGVKVLDSVNITLGAEDELKDAVGLVRPVSVAFEVITGFRLYKSGVVTSDHCGTTPMDVNHAVLAVGYGVEDGVPYWLIKNSWGADWGDEGYFKMEMGKNMCGVATCASYPIVA

SEQ ID NO: 24 - protein, 363 aa, Zea mays
MAHRRIILLLAVAAVAATSAVAAASSGFDDSNPIRPVTDRAASALESTVFAALGRTRDALRFARFAVRYGKSYESAAEVHKRFRIFSESLQLVRSTNRKGLSYRLGYNRFADMSWEEFRATRLGAAQNCSATLTGNHRMRAAAVALPETKDWREDGIVSPVKNQGHCGSCWTFSTTGALEAAYTQATGKPISLSEQQLVDCGFAFNNFGCNGGLPSQAFEYIKYNGGLDTEESYPYQGVNGICKFKNENVGVKVLDSVNITLGAEDELKDAVGLVRPVSVAFEVITGFRLYKSGVYTSDHCGTTPMDVNHAVLAVGYGVEDGVPYWLIKNSWGADWGDEGYFKMEMGKNMCGVATCASYPIVA

SEQ ID NO: 25 - protein, 362 aa, Oryza sativa
MAHRRIILLLAVAAVAATSAVAAASSGFDDSNPIRSVTDHAASALESTVIAALGRTRGALRFARFAVRGHKRYGDAAEVQRRFRIFSESLELVRSTNRRGLPYRLGINRFADMSWEEFQASRLGAAQNCSATLAGNHRMRDAPALPETKDWREDGIVSPVKDQGHCGSCWPFSTTGSLEARYTQATGPPVSLSEQQLADCATRYNNFGCSGGLPSQAFEYIKYNGGLDTEEAYPYTGVNGICHYKPENAGHKVLDSVNITLVAEDELKNAVGLVRPVSVAFQVINGFRMYKSGVYTSDHCGTSPMDVNHAVLAVGYGVENGVPYWLIKNSWGADWGDNGYFTMEMGKNMCGIATCASYPIVA

(19) United States
(12) Patent Application Publication
(10) Pub. No.: US 2003/0070188 A1
(43) Pub. Date: Apr. 10, 2003

(54) Vanillin biosynthetic pathway enzyme from Vanilla planifolia

(76) Inventors: Daphna Havkin-Frenkel, North Brunswick, NJ (US); Andrzej Podstolski, Warsaw (PL); Richard A. Dixon, Ardmore, OK (US)

Correspondence Address: Janet E. Reed, Esq., WOODCOCK WASHBURN LLP, One Liberty Place - 46th Floor, Philadelphia, PA, 19103, US

(21) Appl. No.: 10/087,714

(22) Filed: Feb. 28, 2002

Related U.S. Application Data
(63) Continuation-in-part of application No. 09/462,576, filed on May 25, 2000.
Application No. 09/462,576 is a 371 of international application No. PCT/US98/14895, filed on Jul. 15, 1998.
(60) Provisional application No. 60/052,604, filed on Jul. 15, 1997.
(60) Provisional application No. 60/272,415, filed on Feb. 28, 2001.

(51) Int. Cl. C12P 7/24; A01H 1/00

(52) U.S. Cl. 800/278; 435/147

(57) ABSTRACT

Novel compositions and methods for improving vanillin production in cultured Vanilla planifolia and in intact plants are provided. Transgenic cells and plants having improved vanillin production are also provided. Isolated 4-hydroxybenzaldehyde synthase enzyme and nucleic acids encoding the enzyme are further provided.

### CROSS REFERENCE TO RELATED APPLICATIONS

This application is a continuation-in-part of U.S. application Ser. No. 09/462,576, having a filing date of May 22, 2000, which is a U.S. national application of International Application PCT US98/14895, filed Jul. 15, 1998, which claims benefit of U.S. Provisional Application No. 60/052,604, filed Jul. 15, 1997. This application also claims benefit of U.S. Provisional Application No. 60/272,415, filed Feb. 28, 2001. The entirety of each of the applications mentioned in this paragraph is incorporated by reference herein.

### FIELD OF THE INVENTION

This invention relates to the field of plant genetic engineering to improve agronomic or commercial properties of plants. In particular, this invention provides a novel enzyme and its encoding nucleic acid molecule, isolated from Vanilla planifolia, which is an integral part of the biosynthetic pathway of vanillin.

### BACKGROUND OF THE INVENTION

Vanillin is the principle flavor ingredient in vanilla extract and is also noted as a nutraceutical because of its anti-oxidant and antimicrobial properties. Vanillin can be used as a masking agent for undesirable flavors of other nutraceuticals. Vanilla extract is obtained from cured vanilla beans, the bean-like pod produced by Vanilla planifolia, a tropical climbing orchid.

Vanilla extract is widely used as a flavor by the food and beverage industry, and is used increasingly in perfumes. The U.S. annual consumption of vanilla beans, all of which are imported from foreign countries, is 1,200-1,400 tons, which at a cost of $200 per kg represents a market value in excess of $200 million. By FDA definition, vanillin can be labeled as natural only when it is derived from vanilla beans. Currently, natural vanilla obtained through extraction of vanilla beans as described below, costs between $1,500 and $3,000 per kilogram. Vanillin is also produced by molecular breakage of curcumin, eugenol or piperin at a cost of $100-700/kg. However, vanillin produced by this method can be labeled as a natural flavor only in non-vanilla flavors. Vanillin chemically synthesized from guaiacol, and to a lesser extent from lignin, is consumed at a rate of about 800-1000 tons per year in the United States for the food and beverage industry, at a cost of production of about $10-15/kg.

Currently, natural vanilla extract produced from vanilla beans is the most desirable form of vanilla, due to the recent demand for natural food ingredients. The areas of the world capable of supporting vanilla cultivation are limited, due to its requirement for a warm, moist and tropical climate with frequent, but not excessive rain, and moderate sunlight. The primary growing region for vanilla is around the Indian Ocean, in Madagascar, Comoros, Reunion and Indonesia.

The production of vanilla beans is a lengthy process that is highly dependent on suitable soil and weather conditions. Beans (pod-like fruit) are produced after 4-5 years of cultivation. Flowers must be hand-pollinated, and fruit production takes about 8-10 months. The characteristic flavor and aroma develops in the fruit after a process called “curing,” lasting an additional 3-6 months. For a complete review of the vanilla growing and curing process, see D. Havkin Frenkel & R. Dorn, Vanilla, Chapter 4 in Spices: Flavor Chemistry and Antioxidant Properties, (Eds. Risch & Ho), American Chemical Society, Washington, 1997.

Interest has focused recently on plant cell and tissue culture as an approach to control quality and yield of vanilla production and to solve some of the agronomic problems associated with growing vanilla. Plant tissue culture should be useful for three objectives: (1) micropropagation of vanilla plants; (2) production of vanillin and other secondary products associated with vanilla flavor; and (3) improving production of vanillin in culture or in intact plants by elucidating and manipulating the biosynthetic pathways of vanillin and other flavor compounds. In connection with this last objective, efforts have been made to commercialize production of vanillin, the most valuable component of vanilla, by using plant cell culture. However, these efforts have not resulted in economically significant amounts of vanillin production, perhaps due in part to the heretofore incomplete understanding of the vanillin biosynthetic pathway.

From the foregoing, it can be seen that improvement of vanillin production, either in tissue culture or in intact plants, would be of significant agronomic and economic advantage. Accordingly, it would be useful to provide means for obtaining high yields of vanillin from cultured cells and tissues and to improve vanillin production in intact vanilla plants. It would also be useful to identify and isolate novel enzymes in the vanillin biosynthetic pathway of V. planifolia, and their encoding nucleic acid molecules, for use in enhancing vanillin production in cultured cells or in intact plants.

### SUMMARY OF THE INVENTION

Novel compositions and methods for improving vanillin production in cultured Vanilla planifolia and in intact plants are provided. These cultures and plants are expected to be of significant agronomic and economic value.

According to one aspect of the invention, a method for improving production of vanillin in cultured Vanilla planifolia is provided. The method comprises supplementing the culture with a compound selected from the group consisting of malic acid, 3,4-dihydroxybenzaldehyde, citric acid, pyruvic acid, oxaloacetic acid, succinic acid, glycosylated lysozyme, and any combination thereof, in an amount effective to improve the vanillin production as compared with cultures not supplemented with the compound.

In preferred embodiments of the invention, the tissue culture is an embryo culture. In another preferred embodiment, the culture is supplemented with malic acid at a concentration of between about 0.01% and 5% by weight of the culture medium. In another preferred embodiment, the culture is supplemented with 3,4-dihyrdoxybenzaldehyde at a concentration of between about 0.1 and 5 mM. In another embodiment, the culture is supplemented with about 0.01 to about 5% by weight of a compound selected from the group consisting of succinic acid, oxaloacetic acid, citric acid and pyruvic acid. In yet another embodiment, the culture is supplemented with about 1 to about 100 μg/ml of a glycosylated lysozyme elicitor.

According to another aspect of the invention, cultured Vanilla planifolia cells, produced by the aforementioned method, are provided. These cells preferably produce at least twice as much vanillin as equivalent cultured cells not supplemented with the listed compounds.

In an particularly preferred embodiment the cells produce at least ten times, and most preferably 50 to 100 times, as much vanillin as equivalent cultured cells not supplemented with the compounds.

According to another aspect of the invention, a second method for improving production of vanillin in cultured Vanilla planifolia is provided. This method comprises subjecting the culture to a stress condition selected from the group consisting of heat stress and mechanical shear stress, in an amount and for a time effective to improve the vanillin production as compared with cultures not subjected to the stress condition. In a preferred embodiment, the heat stress comprises maintaining the cultures between about 33 and 37° C. for between three and seven days. In another embodiment, the mechanical shear stress is imposed by placing the cultures in an impeller-driven incubator, under conditions whereby the shear stress is caused.

Cultured Vanilla planifolia cells produced by the aforementioned method are also provided. In a preferred embodiment, these cells produce at least twice as much vanillin as equivalent cultured cells not subjected to the stress.

According to another aspect of the invention, a method for improving vanillin production in Vanilla planifolia, is provided, which comprises genetically engineering the Vanilla planifolia to overproduce one or more enzymes associated with one or more steps of vanillin biosynthesis in the Vanilla planifolia. The steps are selected from the group consisting of: chain shortening of p-coumaric acid (sometimes referred to herein as 4-coumaric acid) to produce p-hydroxybenzaldehyde (sometimes referred to as 4-hydroxybenzaldehyde) and acetic acid; chain shortening of ferulic acid to vanillin; hydroxylation of p-hydroxybenzyl alcohol to 3,4-dihydroxybenzyl alcohol or aldehyde; and methylation of 3,4-dihydroxybenzaldehyde to vanillin. The enzymes preferably are selected from the group consisting of: at least one p-hydroxybenzaldehyde synthase (sometimes referred to as p-coumaric acid chain shortening enzyme, or 4-hydroxybenzaldehyde synthase (4HBS)); at least one cytochrome P450 monooxygenase; and at least one methyl transferase. In some embodiments, one or more of the enzymes can utilize as preferred substrates molecules whoich are esterified.

In another aspect of the invention, the enzyme selected for overexpression is an isolated V. planifolia 4-hydroxybenzaldehyde synthase (4HBS), which has been cloned and sequenced in accordance with the present invention. An isolated nucleic acid sequence encoding this enzyme is set forth herein as SEQ ID NO:1, and its encoded protein is set forth herein as SEQ ID NO:2.

In another aspect of the invention a 4-HBS enzyme (sometimes referred to herein as chain shortening enzyme (CSE)) is provided. The enzyme has the ability to convert 4-coumaric acid to 4-hydroxybenzaldehyde and acetic acid. The enzyme in a presently preferred embodiment has enhanced activity in the presence of thiol compounds or in an in vivo reducing environment. In another embodiment, no cofactors are required. The enzyme is isolated from natural sources in some embodiments or can be generated by a protein expression system using the nucleic acids of the invention in other embodiments. In a preferred embodiment, the enzyme is a multimeric enzyme in one embodiment, with the subunits having sequence homology to known cysteine proteases.

In one embodiment of the aforementioned method of improving vanillin production, the genetically engineered Vanilla planifolia is a cell or tissue culture. In another embodiment, it is a whole plant. Genetically engineered Vanilla planifolia cells or plants produced by the aforementioned method are also provided. These cells or plants preferably produce at least twice as much vanillin as does an equivalent cell which is not comparably genetically engineered.

According to yet another aspect of the invention, a method for improving vanillin accumulation in cell or tissue culture of Vanilla planifolia is provided, which comprises inhibiting production or activity of vanillyl alcohol dehydrogenase in cells comprising the cell or tissue culture, the inhibition resulting in the improved vanillin accumulation. In one embodiment, the inhibiting comprises genetically engineering the cells to inhibit expression of a gene encoding the vanillyl alcohol dehydrogenase. In another embodiment, the inhibiting comprises treating the culture with an inhibitor of vanillyl dehydrogenase activity. Cultures produced by the aforementioned method are also provided.

According to still another aspect of the present invention, a method for improving vanillin production and accumulation in a Vanilla planifolia cell or tissue culture is provided, which comprises: (a) genetically engineering the Vanilla planifolia to overproduce one or more enzymes associated with one or more steps of vanillin biosynthesis in the Vanilla planifolia, the steps selected from the group consisting of: generally modifying specific groups of esters, for example, esters of shikimic acid, to form vanillin or preferred precursors of vanillin; more specifically, chain shortening of 4-coumaric acid to 4-hydroxybenzaldehyde; chain shortening of ferulic acid to vanillin; hydroxylation of p-hydroxybenzyl alcohol, or corresponding aldehydes or esters, to 3,4-dihydroxybenzyl alcohol or aldehyde; and methylation of 3,4-dihydroxybenzaldehyde to vanillin, thereby resulting in the improved vanillin production; and (b) inhibiting production or activity of vanillyl alcohol dehydrogenase in cells of the culture, thereby resulting in the improved vanillin accumulation. A Vanilla planifolia cell or tissue culture produced by the aforementioned method is also provided.

Additional features and advantages of the present invention will be understood by reference to the drawings, detailed description and examples that follow.

### BRIEF DESCRIPTION OF THE DRAWINGS

FIG. 1. Schematic diagram showing the biosynthetic pathway of vanillin in Vanilla planifolia.

FIG. 2. Graph showing the conversion of p-coumaric acid to p-hydroxybenzaldehyde as catalyzed by p-hydroxybenzaldehyde synthase in V. planifolia embryo culture.

FIG. 3. Graph showing uptake of exogenously added vanillin and its transformation to vanillyl alcohol by vanillyl alcohol dehydrogenase in V. planifolia embryo culture. V(T)=vanillin in tissue; V(M)=vanillin in medium; VAL(T)=vanillyl alcohol in tissue; VAL(M)=vanillyl alcohol in medium.

FIG. 4. Graph showing coumaric acid uptake from culture media containing recombinant cells expressing V. planifolia p-coumaric acid chain shortening enzyme (p-hydroxybenzaldehyde synthase). I=induced with methanol; U=uninduced; +=culture fed with coumaric acid.

FIG. 5. Graph showing p-hydroxybenzaldehyde (BA)-forming activity in recombinant cells expressing V. planifolia p-coumaric acid chain shortening enzyme (p-hydroxybenzaldehyde synthase). I=induced with methanol; U=uninduced; +=cells fed with coumaric acid; −=cells not fed with coumaric acid.

FIG. 6. Comparision of amino acid sequence of CSE (4HBS) with those of other cysteine proteases. Arrows indicate the presence of common features: vacuole sorting signal, cleavage site for proteolytic processing, and active site residues.

FIG. 7. CSE activity is selectively immunoprecipitated by the antibody to the 28 kDa protein. The antibody to the 28 kDa protein expressed in E. coli immunoprecipitates the CSE activity from V. planifolia culture extracts. Incubation with preimmune and immune serum. X-axis represents the volume of culture extract; y-axis represents the production of p-hydroxybenzaldehyde.

FIG. 8. The distribution of the 4HBS protein in V. planifolia tissues. Tissues: St=stem, L=Leaf, B=Bean, Em=Embryo tissues. The antibody to the protein expressed in E. coli detects the 28 kDa protein (Lanes 5-8) in a Western blot. Bean tissue contains highest amount of 4HBS (lane 7). Root not shown. Preimmune serum does not detect any V. planifolia proteins (lanes 1-4).

FIG. 9. In-gel protease activity of 4HBS. The 4HBS (CSE) from V. planifolia extracts does not display any measurable protease activity. In-gel assay performed as described in text. Tissue abbreviations as in FIG. 8. Standard proteases tested were chymotrypsin and collagenase (lane 1). Substrate was gelatin as described in the text.

FIG. 10. Tissue-specific activity of 4HBS in V. planifolia. Specific activity of 4HBS from tissues of V. planifolia. Embryo, root and bean tissues show the highest specific activity as determined by the accumulation of the benzaldehyde product.

### DETAILED DESCRIPTION OF THE INVENTION

I. Definitions

Various terms relating to the biological molecules of the present invention are used hereinabove and also throughout the specification and claims. The terms “substantially the same,” “percent similarity” and “percent identity” are defined in detail below.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule.

With respect to RNA molecules of the invention the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

With respect to oligonucleotides or hybridization generally, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “promoter region” refers to the 5′ regulatory regions of a gene.

The term “reporter gene” refers to genetic sequences which may be operably linked to a promoter region forming a transgene, such that expression of the reporter gene coding region is regulated by the promoter and expression of the transgene is readily assayed.

The term “selectable marker gene” refers to a gene product that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

The term “DNA construct” refers to genetic sequence used to transform plants and generate progeny transgenic plants. These constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

With reference to specific molecules, substrates, and intermediates referred to throughout the specification, it is understood that such molecules may be present in alternate chemical forms, particularly in planta. For example substrates may be present as various esters. Such esters are preferably cleavable in 1 M NaOH. Certain alternate forms may be normal substrates for the enzymes of the present invention, particularly in planta, and as such are contemplated to be included within the meaning of the terms where used.

II. Description

In an effort to obtain commercially feasible tissue culture yields of vanillin and related compounds, the inventors have now elucidated the biosynthetic pathway by which these compounds are produced out of several possible pathways which have been proposed, and have determined the rate-limiting step in the biosynthesis. The important discovery of the correct pathway, and the rate limiting step in particular, has contributed to the development of high-yield tissue culture for vanillin production.

Another important feature of the present invention is the use of embryo cultures of vanilla plants for the purpose of producing vanillin at an economically feasible level. Embryo culture of Vanilla planifolia is described in detail in Example 1.

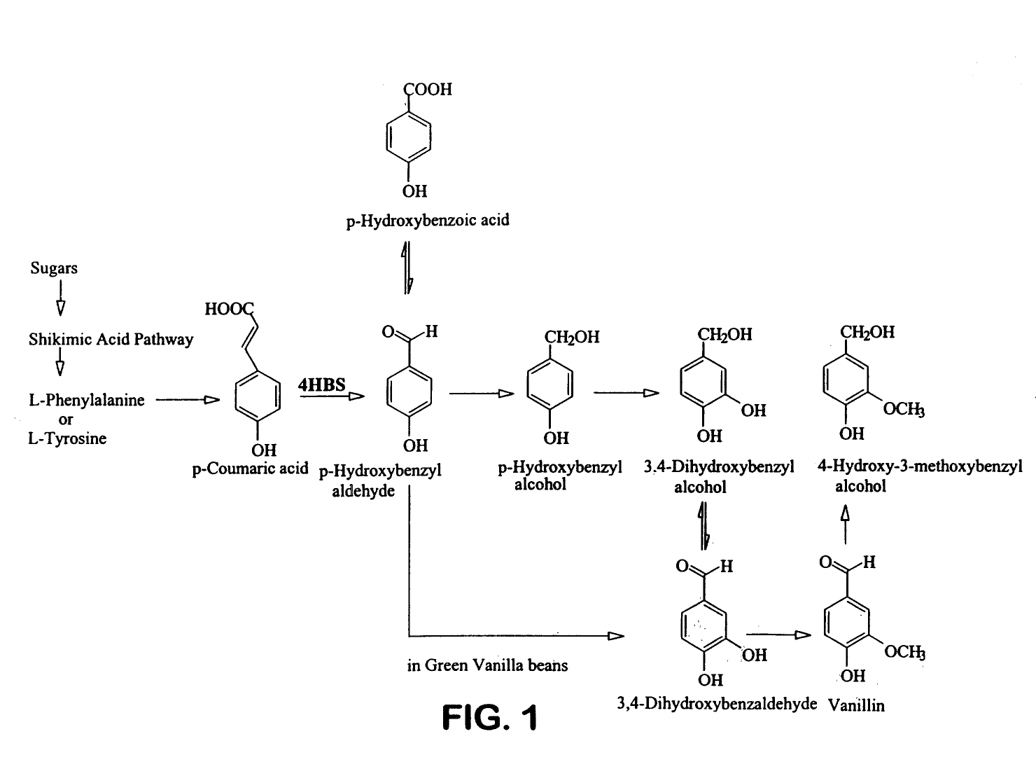

The vanillin biosynthetic pathway is shown in FIG. 1 and described in detail in Example 2. As can be seen from FIG. 1, p-coumaric acid is produced from L-phenylalanine via the shikimic acid pathway. The first key step in the pathway is the chain shortening of p-coumaric acid to form p-hydroxybenzyaldehyde, then p-hydroxybenzyl alcohol. The next key step is the hydroxylation of p-hydroxybenzyl alcohol to 3,4-dihydroxybenzyl alcohol, then 3,4-dihydroxybenzaldehyde (sometimes referred to herein as “proaldehyde”). This is believed to be the rate limiting step in the pathway. Proaldehyde is next methylated to form vanillin (3-methoxy-4-hydroxybenzaldehyde). In some embodiments, one or more ethyl transferases are involved in the pathway and one or more of the intermediates in the pathway are in form of esters. In cultured cells, much of the vanillin produced is reduced to vanillyl alcohol, which is a detrimental occurrence inasmuch as it depletes the culture of accumulated vanillin.

The enzymes involved in the vanillin biosynthetic pathway are believed to be the following. The chain shortening of p-coumaric acid to form p-hydroxybenzaldahyde is catalyzed by at least one chain-shortening enzyme, sometimes referred to herein as 4-hydroxybenzaldahyde synthase (4HBS). The partial purification and characterization of a 4HBS from V. planifolia is described in Example 6, while further purification and characterization is presented in Example 12.

A nucleic acid molecule encoding a 4HBS (also referred to as 4-hydroxybenzaldahyde synthase or p-hydroxybenzaldehyde synthase) has now been isolated from V. planifolia. Its sequence is set forth herein as SEQ ID NO:1. The amino acid sequence of its encoded polypeptide is set forth herein as SEQ ID NO:2.

The 4HBS-encoding nucleic acid has been successfully expressed in E. coli and yeast cells, as well as the creeping bentgrass (Agrostis palustris Huds.) and Arabidipsis thaliana. The cloning and expression of the 4HBS in yeast, and a demonstration of activity in yeast, is described in detail in Example 10.

The enzyme catalyzing the rate-limiting hydroxylation of p-hydroxybenzyl alcohol to 3,4-dihydroxybenzyl alcohol is believed to be a cytochrome P450 monooxygenase. Strategies for cloning the gene(s) encoding the enzyme(s) are described in greater detail below and in Example 7.

The enzyme catalyzing the methylation of 3,4-dihydroxybenzaldehyde to vanillin has been determined to be an O-methyltransferase. This methyl transferase was purified from cultured vanilla cells or from intact plants, according to one of several methods available in the art for purifying O-methyl transferase. The enzyme has been purified, sequenced and the Km for over 15 substrates was determined. In a one embodiment, it is purified according to the method of Edwards & Dixon, Arch. Biochem. Biophys. 287: 372-379, 1991.

The substrates recognized by either or both of the cytochrome P450 and the O-methyl transferase in some embodiments can include C3-C1 benzoic acids and aldehyde substituents.

The enzyme catalyzing the conversion of vanillin to vanillyl alcohol has been determined to be an alcohol dehydrogenase, which the inventors have named vanillyl alcohol dehydrogenase (VAD). The purification of VAD from cultured cells of V. planifolia and its characterization are described in Example 8.

In the present invention, two general approaches are used to improve vanillin production in cultured cells and, in some instances, in intact vanilla plants. The first approach employs manipulation of tissue culture conditions to increase vanillin accumulation in cultured cells. The second approach employs genetic manipulation of the vanillin biosynthetic pathway by up-regulating or down-regulating, as appropriate, enzymes involved in the vanillin biosynthetic pathway or in the conversion of vanillin to vanillyl alcohol. These approaches are described below.

A. Improving Vanillin Production in Tissue Culture by Manipulation of Culture Conditions

It has been discovered in accordance with the invention that addition of certain “elicitor” compounds provides a surprisingly high yield of vanillin and related compounds in plant tissue culture, particularly embryo culture. These elicitor compounds include malic acid, citric acid, succinic acid, pyruvic acid and oxaloacetic acid. No method heretofore described has employed these compounds in plant tissue culture to stimulate production of vanillin and similar compounds.

Malic acid is especially successful in this respect. The use of malic acid in an amount effective to increase production of vanillin and related compounds in plant tissue is an important part of the present invention. Malic acid may be used with any type of plant tissue, under any form of cultivation or in any conditions known for plant tissue culture. For instance, Table 6 in Example 5 shows that 3% malic acid used in elicitation of embryo cultures increases vanillin yield from 5 to 72 mg/100 g tissue. Citric acid, succinic acid, pyruvic acid and oxaloacetic acid may also be used in amounts effective to increase yields of vanillin and related compounds at least two-three-fold in plant tissue culture, such as at about 0.1% to about 5.0%, preferably from about 0.5 to 3.0%, and otherwise as discussed for malic acid here and in the

### EXAMPLES

Another useful elicitor of vanillin production in cultured vanilla is the glycosylated lysozyme protein elicitor described in U.S. Pat. No. 5,552,307 to Kessler et al. As shown in Example 5, treatment with this elicitor more than doubles the amount of vanillin produced in cultured vanilla cells.

Another elicitor of vanillin production in cultured cells is heat stress, i.e. placing the cultures at 33-37° C. for an extended period of time. Heat stress of this nature has been found to increase production of vanillin and related compounds in cultured cells by at least 2-3-fold. Similarly, shear stress, as described in greater detail in the examples, increases production of vanillin and related compounds in cultured cells by at least 2-3 fold.

Also in accordance with the present invention, vanillin production in cultured cells may be improved by feeding the cultures with an excess of any of the precursors or intermediates in the vanillin biosynthetic pathway. For instance, proaldehyde (3,4-dihydroxybenzaldahyde, the immediate precursor of vanillin) can be used with any type of plant tissue under any form of cultivation or any conditions known for plant tissue culture to stimulate vanillin synthesis. A proaldehyde concentration of 0.1 to 5.0 mM is especially useful. Examples of specific conditions for addition of proaldehyde are set forth in Example 3. Furthermore, Example 3 also describes that precursors can be fed to intact green vanilla beans to improve vanillin production in the beans.

B. Improving Vanillin Production in Tissue Culture and Intact Plants by Manipulation of Enzymes of the Vanillin Biosynthetic Pathway

Manipulation of the enzymes involved in the vanillin biosynthetic pathway is another approach used in accordance with this invention to improve vanillin production in vanilla tissue culture and in intact plants. As discussed below and in the examples, the inventors have either isolated these enzymes or devised means for their isolation using standard methodologies known in the art, as described in greater detail in the Examples. These enzymes may be added to or inhibited in plant cultures directly, or plant tissues may be engineered for altered expression of the genes encoding the enzymes, by one of several methods as described below.

The first key enzyme in the vanillin biosynthetic pathway is the enzyme referred to herein as the “chain shortening enzyme”, p-hydroxybenzaldehyde synthase, 4-hydroxybenzaldehyde synthase (4HBS), which catalyzes the conversion of 4-coumaric acid to 4-hydroxybenzaldehyde. Though the chain shortening enzyme may be referred to in the singular, it is possible that this activity is performed by more than one enzyme. A nucleic acid molecule (SEQ ID NO:1) that encodes a polypeptide (SEQ ID NO:2) having this chain shortening activity has been isolated in accordance with the present invention. The conversion of 4-coumaric acid to 4-hydroxybenzaldehyde is not considered to be the rate-limiting step in vanillin biosynthesis in cultured cells. However, this reaction may play a more important rate-controlling function in intact vanilla beans. In either case, it is believed that up-regulation or some other form of supplementation of this enzyme will enhance vanillin production in cultured cells and in intact plants.

The present invention provides a gene encoding a 4HBS enzyme activity from V. planifolia. The sequence of the gene is set forth as SEQ ID NO:1. The enzyme encoded by the gene has novel properties. The enzyme is initially synthesized as an immature 39 kDa precursor (as encoded by SEQ ID NO:1) but is subsequently posttranslationally processed to its mature 28 kDa size. The mature 28Kda protein, when expressed in E. coli, was catalytically inactive but immunogenically active. When expressed in yeast, the protein can be properly post-translationally processed to a catalytically-active, mature size protein. In some embodiments, the protein is expressed in other plants, wherein it retains it catalytic activity.

In one embodiment, the invention provides a catalytically-active 4HBS. The enzyme in a presently preferred embodiment is optimally active in the presence of a thiol compound, such as DTT, or in an in vivo reducing environment with biologically-active thiol donors. The 4HBS has a high degree of amino acid sequence homology with several known cysteine proteases, particularly plant cysteine proteases, more particularly to plant cysteine proteases which are induced during senescence, even more particularly to the mature portion of plant cysteine proteases which are induced during senescence. The enzyme however does not demonstrate protease activity with gelatin as a potential substrate nor with other substrates tested to date.

The enzyme can be purified from various sources by the sequential steps of ammonium sulphate fractionation, hydrophobic interaction chromatography, ion exchange chromatography and size exclusion chromatography. The enzyme in one embodiment forms multimers which associate with each other in an active form. The enzyme preferably requires no cofactors for catalysis.

In preferred embodiments, the enzyme activity is expressed in stems, leaves, roots and pods of V. planifolia, and most fully expressed in the roots and mature pods. Cells which accumulate vanillin are presently preferred sources of the enzyme activity. The enzyme also is produced in cells genetically modified with nucleic acid molecules of the invention. The enzyme activity can be generated through genetic means in yeast, for example, the activity is expressed in Pichia pastoris using a pPIC19 expression vector in one embodiment. The enzyme activity is expressed in higher plants, for example the monocot, creeping bentgrass.

Another chain shortening enzyme that is expected to be useful for practice of the invention is the chain-shortening enzyme(s) that catalyze the conversion of ferulic acid to vanillin. These enzymes may be used in conjunction with a growth medium containing ferulic acid, to stimulate production of vanillin.

The next key enzyme in the vanillin biosynthetic pathway is the oxygenase that catalyzes hydroxylation of p-hydroxybenzyl alcohol to 3,4-dihydroxybenzyl alcohol. This enzyme is believed to be a cytochrome P450 monooxygenase, and this step is believed to be the rate-limiting step in the vanillin biosynthetic pathway in cultured cells. For these reasons, up-regulation or some other form of supplementation of this enzyme in cultured cells and in intact plants.

The next key enzyme in the vanillin biosynthetic pathway is the methyl transferase that catalyzes the conversion of 3,4-dihydroxybenzaldehyde (proaldehyde) to vanillin. In cultured vanilla or in intact plants, regulation of this enzyme activity, for example up-regulation or some form of supplementation of this enzyme augments vanillin accumulation in some embodiments.

The next key enzyme, vanillyl alcohol dehydrogenase (VAD) actually catalyzes the destruction of vanillin rather than its synthesis. VAD is a novel enzyme found in vanilla tissue culture, but not in any significant amount in vanilla beans. The inventors have found that vanilla beans produce and accumulate vanillin as a final product, whereas in tissue culture, vanillin is produced but is converted by VAD and stored as vanillyl alcohol. Accordingly, down-regulation of VAD in cultured cells is a key feature of improved vanillin production from cultured vanilla. In practice, vanillin is found mostly as a glucoside, as is much of 4-hydroxybenzyl alcohol, while many other intermediates are found as esters which can be hydrolyzed with 1 N NaOH. In general, when intermediates are present as esters they are still active in the present pathways.

In some instances it may be possible to add one or more of the above-listed enzymes directly to a vanilla cell or tissue culture, to enhance the biosynthetic activity of endogenous enzymes and increase vanillin production. This may be useful, for example, where a particular activity is rate limiting in the desired pathway. However, it is preferred for practice of the present invention to augment or reduce activity of one or more of the enzymes by internal manipulation; e.g. up-regulation by genetic engineering to increase transcription or translation of endogenous genes or transgenes, or down-regulation by expression of antisense molecules or antibodies that specifically bind to genes encoding the enzymes or to the enzymes themselves, respectively, or by expression of non-functional mutants, or by an overexpression/co-suppression effect.

In order to genetically manipulate the vanillin biosynthetic pathway, it is necessary to have in hand nucleic acid molecules that encode selected key enzymes of that pathway. The availability of purified or semi-purified biosynthetic pathway enzymes, as described in greater detail in the Examples, enables obtaining their encoding nucleic acid sequences by a variety of methods known in the art. Such methods can be found in general references such as Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”).

In a preferred embodiment, antibodies immunologically specific for a selected key enzyme in the vanillin biosynthetic pathway are produced, then used to screen a cDNA library made either from cultured vanilla cells or from intact plants. In an alternative embodiment, purified enzymes are partially or fully sequenced, and a set of degenerate oligonucleotide probes is produced, which encodes part or all of the sequence. These probes may be used to screen either a genomic or cDNA library by standard means or via PCR amplification.

One common formula for calculating the stringency conditions required to achieve hybridization between nucleic acid molecules of a specified sequence homology (Sambrook et al., 1989):

Tₘ=81.5° C.+16.6Log[Na+]+0.41(% G+C)−0.63 (% formamide)−600/#bp in duplex

As an illustration of the above formula, using [N+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

Nucleic acids encoding vanillin biosynthetic pathway enzymes, obtained in accordance with the present invention, may be maintained as DNA in any convenient cloning vector. In a preferred embodiment, clones are maintained in plasmid cloning/expression vector, such as pGEM-T (Promega Biotech, Madison, Wis.) or pBluescript (Stratagene, La Jolla, Calif.), either of which is propagated in a suitable E. coli host cell.

Specific cloning strategies for the various key enzymes of the vanillin biosynthetic pathway are set forth in the Examples. Once cloned DNA is obtained, it may be used to genetically manipulate the vanillin biosynthetic pathway by enhancing or inhibiting, as appropriate, selected enzymes of the pathway.

Transgenic plants can be generated using standard plant transformation methods known to those skilled in the art. These include, but are not limited to, Agrobacterium vectors, PEG treatment of protoplasts, biolistic DNA delivery, UV laser microbeam, gemini virus vectors, calcium phosphate treatment of protoplasts, electroporation of isolated protoplasts, agitation of cell suspensions with microbeads coated with the transforming DNA, direct DNA uptake, liposome-mediated DNA uptake, and the like. Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratory Manual (Maliga, Klessig, Cashmore, Gruissem & Varner, eds., 1994).

The method of transformation depends upon the plant to be transformed. The biolistic DNA delivery method is useful for nuclear transformation, and is preferred for practice of the present invention. Transformation of V. planifolia using the biolistic method is described in detail in Example 9. In another embodiment of the invention, Agrobacterium vectors, particularly binary vectors such as BIN19, are used for transformation of plant nuclei.

Nucleic acids encoding vanillin biosynthetic enzymes may be placed under a powerful constitutive promoter, such as the rice actin promoter or the maize ubiquitin promoter, both of which are particularly useful for gene expression in monocots. Other constitutive promoters that may also prove useful include the Cauliflower Mosaic Virus (CaMV) 35S promoter or the figwort mosaic virus 35S promoter. Alternatively, transgenic plants expressing one or more of the genes under an inducible promoter (either their own promoter or a heterologous promoter) are also contemplated to be within the scope of the present invention. Inducible plant promoters include the tetracycline repressor/operator controlled promoter.

Using a biolistic delivery system for transformation, the coding region of interest, under control of a constitutive or inducible promoter as described above, is linked to a nuclear drug resistance marker, such as hygromycin resistance. Biolistic transformation of plant nuclei is accomplished according to the following procedure:

(1) the gene is inserted into a selected vector;

(2) transformation is accomplished by bombardment with DNA-coated microparticles, as described in Example 9;

(3) plant tissue is then transferred onto the selective medium to identify transformed tissue; and

(4) identified transformants are regenerated to intact plants or are maintained as cultured cells.

It should be recognized that the amount of expression, as well as the tissue specificity of expression of the transgenes in transformed plants can vary depending on the position of their insertion into the nuclear genome. Such position effects are well known in the art. For this reason, several nuclear transformants should be regenerated and tested for expression of the transgene.

In some instances, it may be desirable to down-regulate or inhibit expression of endogenous enzymes, such as VAD in cultured V. planifolia. Accordingly, VAD-encoding nucleic acid molecules, or fragments thereof, may also be utilized to control the production of VAD. In one embodiment, full-length VAD gene antisense molecules or antisense oligonucleotides, targeted to specific regions of VAD-encoding mRNA that are critical for translation, are used. The use of antisense molecules to decrease expression levels of a pre-determined gene is known in the art. In a preferred embodiment, antisense molecules are provided in situ by transforming plant cells with a DNA construct which, upon transcription, produces the antisense sequences. Such constructs can be designed to produce full-length or partial antisense sequences.

In another embodiment, overexpression of a VAD-encoding gene is induced to generate a co-suppression effect. This excess expression serves to promote down-regulation of both endogenous and exogenous VAD genes. Alternatively, transgenic plants can be created containing mutations in the region encoding the active site of the enzyme, thereby creating a pool of non-functional enzyme in the plant cells, which competes for substrate (i.e., vanillin), but is unable to catalyze the conversion to the undesired product (vanillyl alcohol).

From the foregoing discussion, it can be seen that genetic manipulation of the enzymes involved in the vanillin biosynthetic pathway will produce engineered plant tissue culture and, if desired, intact plants capable of high yield of vanillin and related compounds of value. This approach, alone or combined with the alternative approach of stimulating vanillin production in cultured cells by supplementation with elicitors or biosynthetic precursors, result in improved production of vanillin from a variety of sources, in accordance with the present invention.

The following specific examples are provided to illustrate embodiments of the invention. They are not intended to limit the scope of the invention in any way.

### EXAMPLE 1

### Protocol for Initiation of Vanilla Embryo Culture

Green vanilla beans (from Indonesia), 2 to 8 months after pollination, were washed with cold water, then with mild detergent and water, and were next held for 30 minutes in a water solution containing 20% bleach and a drop of Tween-80. The beans were then rinsed in sterile water and dried. Seeds from the washed beans were scraped and placed on a petri plate containing solid medium (“G-medium”) as follows.

Gamborg's B-5 basal medium

2% sucrose

vitamins (see attached list)

antibiotics—cefotaxime sodium and vancomycin sodium at 100 mg/l each; optionally, tetracycline or chloramphenicol at 50 mg/l each; 0.8% agar

Beans were dissected transversely or longitudinally and the tissue containing the seeds planted on the agar.

To break seed dormancy and to accelerate germination, we applied the following:

10 ppm ethylene

100% oxygen

1-10 μM urea

1-10 μM abscisic acid

1-100 μM gibberellin

Heat shock (37° C. for 3 hours)

Cold shock (2-3° C. for 48 hours)

After 2 to 6 months, seeds germinated and were transferred to fresh agar medium. When germinating shoots reached about 10 mm, they were dissected in half and transferred to fresh agar medium. This process was repeated every two weeks for three months. The agar-cultured embryo tissue was transferred to liquid G-medium without agar. The liquid culture was maintained on an orbital shaker at 130 rpm. Embryo culture was subcultured every two weeks by collecting embryos on a sieve and dissecting the growing embryos into 2 to 4 pieces, depending on the size.

Some embryos were maintained on solid medium and some were kept on rafts (Sigma, St. Louis, Mo.). All cultures were held in light (80 μE/sec/cm) at 25-28° C.

The protocol for initiation of cell suspensions from embryo cultures of V. planifolia was as follows. Established embryo cultures were transferred from petri plates containing G-medium to the same medium, supplemented with 1 μM 2,4-D, then subcultured for two weeks. After callus was initiated, it was transferred to solid medium containing 0.5 μM 2,4-D. After 2 to 6 additional subcultures, the resulting soft callus was transferred to liquid G-medium with 0.5 μM 2,4-D and maintained as cell suspension.

### EXAMPLE 2

### Scheme for Vanillin Biosynthetic Pathway

A scheme was derived for the vanillin biosynthetic pathway by analysis of metabolites in cultured embryos and by experiments with feeding of precursors and intermediates. This scheme is shown in FIG. 1 and the experiments are described below.

Embryo cultures of Vanilla planifolia were established as described in Example 1. The procedures used to extract phenolics from the cultured embryo cells and to analyze the extracts by high pressure liquid chromatography (HPLC) were as described generally by Havkin-Frenkel et al., Plant Cell, Tissue and Organ Culture 45: 133-136 (1996).

In the extraction procedure, 3 ml of 0.05 M sodium acetate buffer, pH 5.5, was added to about 1 gram of fresh culture. Samples were placed in boiling water for three minutes, then chilled. The cells were next homogenized in a Polytron blender for 1 minute at medium speed. A β-glucosidase solution was added to the homogenized cells to give a final concentration of 0.2%. Each sample was then incubated at 37° C. for 5 hr. Next, 17 ml of 95% ethanol was added, after which incubation at 37° C. was continued for an additional 24 hrs. The extract was then filtered and the ethanol evaporated. The filtrate was extracted twice using ethyl acetate, then extracted twice with ethyl acetate acidified to pH 3 with HCl. The extracts were combined and the ethyl acetate evaporated. The residue was dissolved in 1 ml of acidified methanol and filtered with a 0.45 μm syringe filter for HPLC analysis.

Extractions of metabolites from culture medium was similar to the cell extraction protocol. Five ml of spent medium was incubated in a β-glucosidase solution at 37° C. for 24 hrs. The medium was then extracted with ethyl acetate and the organic portions combined and evaporated, as for the cell extracts.

Metabolite levels were measured with a Hewlett Packard 1090L or a Waters HPLC with a UV detector at 280 nm. The Waters HPLC was also equipped with a diode array detector to confirm the identities of the various intermediates, and the identities of the various intermediates were further confirmed by mass spectrometry. The column was a Supelco C-18 DB column of dimensions 250 mm×4.6 mm and a particle size of 5 μm. The mobile phase contained methanol and water, each of which was acidified with 1.25% acetic acid. The flow rate was 1 ml/min, with a solvent gradient as follows:

| Time (min) | % Water |
|---|---|
| 0-10 | 85 |
| 20-25 | 80 |
| 30-42 | 50 |
| 42-end | 85 |

HPLC analyses of tissue extracts from cultured embryos revealed threshold levels of p-coumaric acid, p-hydroxybenzoic acid and p-hydroxybenzaldehyde; usually high levels of p-hydroxybenzyl alcohol, trace levels of 3,4-dihydroxybenzyl aldehyde (Pro-ald), vanillin and vanillyl alcohol.

It is known that coumaric acid (CA) is derived from the deaminization of phenylalanine or tyrosine. The acid, a C6-C₃ compound, is converted by chain shortening to p-hydroxybenzaldehyde (BA), a C6-C₁ compound. Feeding experiments with CA revealed that exogenously applied CA is immediately converted to BA. We examined if benzoic acid or p-hydroxybenzyl aldehyde may be intermediates in the conversion of CA to BA, but a definitive answer has not yet been reached. However, it is clear that at least one chain shortening enzyme is involved in the conversion from CA to BA, and that this step does not appear to be rate-limiting in cultured cells. However, some evidence indicates that it is the rate-limiting step in intact vanilla beans.

A key juncture in the pathway in cultured cells appears to be the hydroxylation of HBA to 3,4-dihydroxybenzyl aldehyde or alcohol and subsquently, vanillin and vanillyl alcohol. Feeding of 3,4-dihydroxybenzyl aldehyde resulted in the rapid methylation and conversion to vanillin or vanillyl alcohol, indicating that these steps are not limiting. The constraint appears to be in the hydroxylation of HBA, for the following reasons:

1. HBA is usually found in higher levels than other intermediates, suggesting a block in further turnover of the compound.

2. Feeding of CA resulted in the accumulation of HBA but only trace amounts of other compounds.

3. Chemical stresses that induce the enzymatic turnover of HBA resulted in the disappearance of the compound and the simultaneous increase in dihydroxybenzyl alcohol or aldehyde, as well as vanillin and vanillyl alcohol.

4. In tissue homogenates where enzyme and substrate are accessible to each other, HBA was rapidly metabolized to dihydroxybenzyl aldehyde. Feeding the homogenates with HBA increased accumulation of dihydroxybenzyl aldehyde, which was the final product since methylation requires intact tissue or the addition of S-adenosyl methionine (SAM).

5. Cytochrome P450 enzymes are normally inducible enzymes that become active at certain stages of development or differentiation. Since the embryo culture is composed of undifferentiated cells, this explains why Cyt P450 activity is not observed.

Thus, these data suggest unhindered metabolite flux to and from HBA in cultured vanilla embryo cells. Hydroxylation of the compound induced by chemical or genetic means is expected to lead to augmented production of vanillin and related compounds in cultured cells.

### EXAMPLE 3

### Use of Vanilla Tissue Culture for the Bio-Conversion of 3,4-Dihydroxybenzaldehyde to Vanillin and Vanillyl Alcohol

The following procedure was used for the bio-conversion of 3,4-dihydroxybenzaldehyde (“proaldehyde”) to vanillin and vanillyl alcohol in V. planifolia tissue culture. The medium used for cultures was G-medium as described above. Proaldehyde solutions were prepared in G-medium, to final concentrations of 0.01 to 5 mM.

Cultures used for the bio-conversion were (1) clusters, (2) embryo culture, and (3) tissue homogenates of the above cultures. Cultures were of ages ranging from 0 to 1 month old. The cultures were allowed to remain under normal culture conditions for 0 to 15 days. As controls, untreated cultures were extracted and analyzed as described in Example 2.

Proaldehyde at different concentrations was added to the medium, either alone or in combination with the following treatments:

malic acid (0.01-3.0%)

varying pH of the medium

varying ascorbic acid concentration

varying temperatures, including cold and heat.

Bioreactor-grown cultures were used for the bio-conversions. Different kinds of impeller designs were used to increase or decrease shear stress on the cells prior to the addition of proaldehyde. As shown in Example 5, it was found that addition of 1-5 mM proaldehyde increased the production of vanillin/vanillyl alcohol by several hundred fold.

In another experiment, the effect of daily refreshing of the proaldehyde containing culture medium was examined. Cultures were transferred to medium containing 5 mM proaldehyde. Control cultures were left in this medium for the duration of the experiment. For test cultures, the medium was removed daily and replaced with fresh medium containing proaldehyde. Cultures subjected to this daily medium change were improved in their appearance and growth, as compared with cultures remaining in the same medium.

### EXAMPLE 4

### Malic Acid-Induced Vanillin and Vanillyl Alcohol Production

Application of malic acid to vanilla tissue culture induced the production/accumulation of vanillin and vanillyl alcohol. Malic acid in concentrations of 0.1 to 3% was applied as a disodium salt to the growing medium. The culture was maintained for 1 to 15 days, then was extracted as described in Example 2. Results are shown in Table 6 in the following example.

Malic acid was applied to the following: (1) intact roots, (2) intact shoots, (3) embryo cultures, (4) cluster cultures, and (5) cuttings. The age of the cultures were between 0 and 1 month. Malic acid was applied alone or in combination with the following: starvation without sugar (sucrose); shear stress induced by bioreactor impeller; citric acid; varying concentrations of oxygen and ethylene; oxaloacetic acid (sodium salt); ascorbic acid; pyruvic acid; glutamic acid; succinic acid; or salt stress.

Adding proaldehyde for a few days, followed by addition of malic acid, was found to increase production of vanillin and vanillyl alcohol. If sucrose is omitted from the malic acid treatment (i.e. starvation due to lack of sucrose), the onset of vanillyl alcohol production occurs more quickly.

Shear stress had a significant effect on vanillin production. The bioreactor with a marine impeller (5 liters, 110-120 rpm, air speed 250 ml/min) was used to culture embryo and cluster cultures for about 21 days. Addition of malic acid after this time resulted in the highest production of vanillin and vanillyl alcohol.

### EXAMPLE 5

### Results of Selected Feeding Experiments

Results of selected experiments in which precursors or elicitors were added to vanilla cultures are set forth below. These experiments were performed in accordance with the procedures set forth in Examples 1-4. The following abbreviations are used:

CA or PC=p-coumaric acid

HY=p-hydroxybenzoic acid

BA=p-hydroxybenzaldehyde

HBA=p-hydroxybenzyl alcohol

Pro-ald=3,4-dihydroxybenzaldehyde

HMBA and Vn. Alc.=vanillyl alcohol

Vn=vanillin

FA=ferulic acid

CAF=caffeic acid

The table below shows results of experiments in which vanillin precursors were fed to vanilla embryo cultures.

TABLE 1
Feeding Vanilla Embryo Culture with
Vanillin Precursors (mg/100 g dry wt.)

| PRECURSORS | CA | HBA | PRO-ALD | VN ALC | VN |
|---|---|---|---|---|---|
| CONTROL | 108 | 9300 | 13 | 5.3 | 0.01 |
| CA (1 mM) | 220 | 13000 | 10 | 10 | 0.01 |
| FA (2.5 mM) | 136 | 11007 | 4.8 | 23.8 | 11.8 |
| CAF (2 mM) | 189 | 11350 | 21 | 32 | 0.5 |
| BA (1 mM) | 125 | 10305 | 5.8 | 12.3 | 0.015 |
| HBA (1 mM) | 158 | 13100 | 4.6 | 9.6 | 0.001 |
| PRO-ALD (1 mM) | 285 | 8950 | 433 | 286.8 | 16.7 |
| VN.ALC. (2 mM) | 206 | 10350 | 1.1 | 882 | 8.5 |

In an experiment with intact plant material, whole green vanilla beans (6 months post-pollination) were infiltrated with various vanillin precursors. The precursors (1.0 mM each in 0.1 M mannitol) were infiltrated by submerging the beans under vacuum into the solutions for 15 minutes, removing and drying the beans, then measuring amounts of precursors daily, for 5 days. The table below shows the results of one such experiment.

TABLE 2
Feeding green vanilla beans with
Vanillin precursors

| PRECURSOR | CA | BA | HY | HBA | PRO-ALD | Vn | Van. Alc |
|---|---|---|---|---|---|---|---|
| CA | + | | | ++ | ++ | ++ | |
| BA | | ++ | | | | | |
| HY | | | ++ | | | | |
| HBA | −+ | | | ++ | ++ | ++ | |
| PRO-ALD | | | | | ++ | +++ | |
| Vn | | | | | | +++ | |
| Van Alc | | | | | | −+ | +++ |

The table below shows the results of experiments in which Fusarium cell walls were added to vanilla embryo cultures as an elicitor of vanillin production. The results show that Fusarium cell walls stimulate production of various precursors of vanillin, up to the apparently rate-limiting step of HBA to pro-ald.

TABLE 3
Effect of Fusarium Cell Wall on Flavor
Production in Vanilla planifolia Embryo Culture

| | mg/100 g Dry Weight | | | |
|---|---|---|---|---|
| TREATMENT/COMPOUND | HBA | HY | BA | PC |
| Control/No Additions | 3700 | 35 | 65 | 52 |
| 27 mg. dry cell wall | 4300 | 50 | 67 | 127 |
| 50 mg. dry cell wall | 6700 | 128 | 198 | 389 |

Culture conditions: Cells were grown for 2 days at 28° C. at 180 RPM

The table below shows the results of experiments testing the effect of chilling temperature on vanillin precursors in vanilla cluster cultures. These results indicate that chilling stress stimulates production of vanillin precursors, up to the rate-limiting conversion of HBA to pro-ald.

TABLE 4
Effect of Chilling Temperature on Vanillin
Precursors in Vanilla planifolia Cluster Culture

| | mg/100 g Dry Weight | | | |
|---|---|---|---|---|
| | HBA | HY | BA | PC |
| 15 Hrs. at 13° C. | 144.0 | 2.1 | 15.9 | 7.2 |
| 15 Hrs. at 13° C. | | | | |
| 17 Hrs. at 28° C. | 232.0 | 1.8 | 26.4 | 7.4 |
| 15 Hrs. at 13° C. | | | | |
| 7 Days at 28° C. | 586.0 | 7.4 | 52.4 | 15.7 |
| Control | 111.0 | 0.74 | 20.3 | 5.5 |
| 7 Days at 28° C. | | | | |

The table below shows the results of experiments testing the effect of the glycosylated lysozyme elicitor proteins described in U.S. Pat. No. 5,552,307 on vanillin precursors in vanilla embryo cultures. As can be seen, these proteins were effective in stimulating vanillin production in the cultured cells.

TABLE 5
Effect of Elicitor Protein on Vanillin Precursors
in Vanilla planifolia Embryo Cultures

| Treatment/ | mg/100 g Dry Weight | | | | | | |
|---|---|---|---|---|---|---|---|
| Compound | HBA | HMBA | PROALD | HY | BA | VN | CA |
| Control/ | 1990.8 | 38.1 | 9.7 | 76.0 | 77.7 | 3.6 | 151.0 |
| No Add | | | | | | | |
| 30 μg/ml | 20006.3 | 30.7 | 137.0 | 83.4 | 79.7 | 8.4 | 152.0 |
| Elicitor | | | | | | | |
| Protein | | | | | | | |
| Added | | | | | | | |

Each point represents an average of 5 flasks.
Culture conditions: Cells were grown for 7 days at 25° C. at 180 RPM

The table below shows the results of HPLC analysis of intermediary metabolites induced by malic acid elicitation in embryo culture and grown under standard conditions, respectively. Cultures were grown in medium containing 3% malic acid by weight, for 7 days. These results show that malic acid stimulates vanillin production in embryo cultures more than tenfold.

TABLE 6
HPLC Analysis of Intermediary Metabolites
Induced by 3% Malic Acid in Vanilla
planifolia Embryo Culture

| | | | | | | | |
|---|---|---|---|---|---|---|---|
| Growth | | | | | | | |
| conditions | HBA | VN | HMBA | PROALD | BA | HY | PC |

(Percent of Dry Weight)

| | | | | | | | |
|---|---|---|---|---|---|---|---|
| Standard | 11.07 | 0.005 | 0.023 | 0.024 | 0.03 | 0.053 | 0.20 |
| Malic | 3.60 | 0.072 | 0.700 | 0.025 | 0.02 | 0.050 | 0.20 |
| Acid | | | | | | | |

### EXAMPLE 6

### Purification and Characterization of Hydroxybenzaldehyde Synthase from Vanilla planifolia Green Embryo Culture

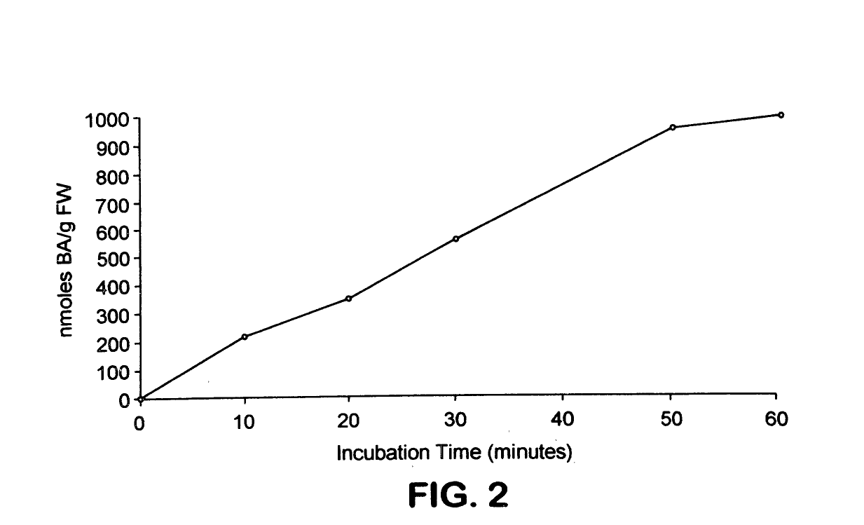

Conversion of p-coumaric acid to p-hydroxybenzyl alcohol in vanilla is catalyzed by at least one chain-shortening enzyme. The rate of conversion as catalyzed by this enzyme is shown in FIG. 2. Characteristics of p-coumaric acid chain shortening enzyme, also referred to as p-hydroxybenzaldehyde synthase, are described in this example. It should also be noted that ferulic acid is converted to vanillin by one or more other chain shortening enzymes, which are believed to be distinct from the p-coumaric acid chain shortening enzyme.

Plant material

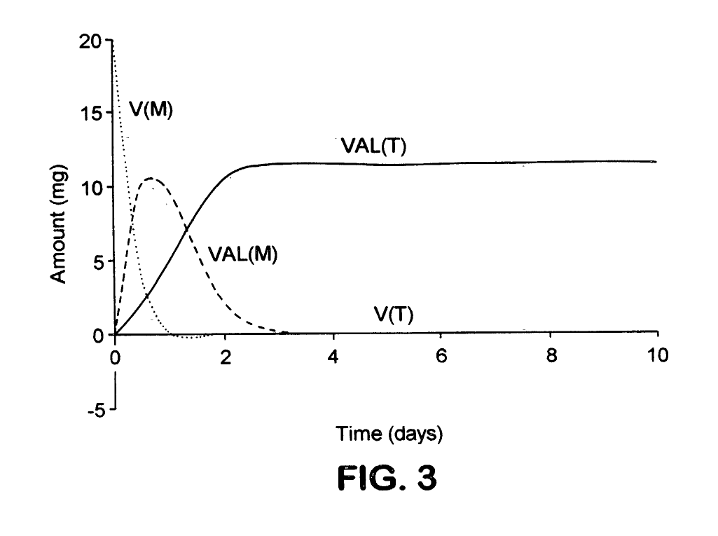

The embryos were cultivated on sterile Gamborg B-liquid medium (3% inoculum,) containing microelements, vitamins, supplemented with 2% of sucrose. The culture was grown at room temperature, under constant illumination (2×OSRAM-DULUX El GLOBE, 100 W each) on rotary shaker (150 rev./min) and subcultured every 2-3 weeks. The conversion of p-coumaric acid to p-hydroxybenzaldehyde as catalyzed by the enzyme(s) is shown in FIG. 3.

Crude Enzyme Extraction

Sterile plant material (2 g) 2-3 weeks after subculture, was homogenized in a cooled Potter glass homogenizer with 4 ml of 0.1 M HEPES buffer, pH 8.0, containing 10 mM DTT. The homogenate was next centrifuged at 4° C. at 15.000× g for 15 min. Resultant supernatant (4 ml) was filtered through Sephadex G-25 column (void volume 3-4 ml.) Equilibrated with Tris/HCL buffer pH 7, containing 10 mM DTT. The column was washed with the same buffer and 2 ml fraction following the void volume was collected. This fraction was used as the crude enzyme source.

The crude enzyme was subjected to SDS polyacrylamide gel electrophoresis. A major band was observed at about 29 kDa.

Determination of the Enzyme Activity

The p-hydroxyaldehyde synthase activity was determined in the following mixture:

| | |
|---|---|
| crude enzyme extract | 10 μ |
| substrate | 100 μl (1.8 mM |
| | p-coumarate in 0.1 M |
| | Tris/HCl, pH 8.0 |
| | containing 10 mM DTT) |

The mixture was incubated for 10-60 min (for longer incubation times, the activity was not proportional with time) at 35° C. and next, the reaction stopped by addition of 200 μl of acidified methanol (10% acetic acid in methanol). The slurry was passed through 0.45 μm filter and the filtrate (50 μl) injected into HPLC Bio-sil 18 HL 90-5 column (Reversed phase, 250 mm×4.6 mm). The HPLC column mobile phase was methanol: water (15:85) acidified with acetic acid (1.25%) at flow rate 1 ml/min. The eluate was monitored at 280 nm and retention time of the reaction product was compared to retention time of the standard compounds (benzaldehyde, p-hydroxybenzaldehyde, vanillin, protocatechuic aldehyde, p-coumaric acid, caffeic acid, ferulic acid, coniferyl aldehyde). The results were quantified using LKB Bromma 2221 Integrator.

TABLE 7
Enzymatic Activity of Crude Preparation of
p-hydroxybenzaldehyde synthase from
V. planifolia

| Incubation time | activity (nmoles of P-HO- |
|---|---|
| (min) | benzaldehyde/g. fr. wt./hr) |
| 10 | 215 |
| 20 | 342 |
| 50 | 946 |
| 60 | 984 |
| 120 | 1022 |

Optimum pH

Extraction efficiency was checked for pH condition from 3 to 9 using citrate and Tris buffers. A broad optimum was found with maximum at pH 8.0. The enzyme pH optimum activity was located between pH 7 and pH 9 and corresponded to optimum of the enzyme extraction.

TABLE 8
Effect of pH of Extraction on Enzymatic Activity

| pH of extraction | activity (nmoles/gfw/h) |
|---|---|
| 3 | 199 |
| 4 | 214 |
| 5 | 258 |
| 6 | 306 |
| 8 | 350 |
| 9 | 205 |

TABLE 9
Effect of pH of Reaction on Enzymatic Activity

| pH of reaction | activity (nmoles/gfw/h) |
|---|---|
| 4 | 29 |
| 5 | 29 |
| 7 | 171 |
| 8 | 548 |
| 9 | 479 |

Stability of the Crude Enzyme Preparation

Samples of the G-25 Sephadex filtered enzyme were stored up to 11 days at 5° C. and frozen at minus 17° C.:

TABLE 10
Stability of Crude Enzyme Preparation

| | |
|---|---|
| 5° C. | −17° C. |

| | |
|---|---|
| Days of storage | % of activity |

| | | |
|---|---|---|
| 0 | 100 | 100 |
| 1 | 55 | 60 |
| 4 | 30 | 41 |
| 6 | 27 | 34 |
| 11 | 0 | 20 |

Ammonium Sulfate Fractionation

HEPES pH 8.0 enzyme extract was subjected to ammionium sulfate fractionation (Salt grinded into fine powder, ice bath).

TABLE 11
Activity in Various Ammonium Sulfate Fractions

| Amm. Sulf. | Protein | total activity | sp. activity |
|---|---|---|---|
| % saturation | content (μg) | (nmol/h) | (mnol/mg/hr) |
| control | 7100 | 7800 | 1098 |
| 0-30 | 4800 | 465 | 97 |
| 30-60 | 450 | 4710 | 10466 |
| 60-95 | 100 | 3510 | 35100 |

These results demonstrate no loss in recovery of total activity and about 35× purification in fraction precipitated between 60 and 95% ammonium sulfate saturation.

Substrate Specificity

As a potential substrates for chain shortening enzyme the following compounds were tested:

TABLE 12
Substrate Specificity of p-hydroxybenzaldehyde Synthetase

| Compound | Expected product | Result |
|---|---|---|
| t-cinnamic acid | benzaldehyde | negative |
| p-coumaric acid | p-HO-benzaldehyde | positive |
| caffeic acid | protocatechuic | negative |
| | aldehyde | |
| ferulic acid | vanillin | negative |
| 4-OH-3-methoxy cinnamyl aldehyde | vanillin? | Negative |
| (also known as coniferyl aldehyde) | | |

These results indicate very high specificity of the tested enzyme towards p-coumaric acid.

### EXAMPLE 7

### Strategies for Cloning a cDNA Encoding the Cyt P450 Monooxygenase that Catalyzes the Rate-Limiting Step in Vanillin Biosynthesis

I. PCR-Based Method

One object of the present invention is the cloning of the cytochrome P450 that catalyzes the 3-hydroxylation of p-hydroxybenzyl alcohol. Although the activity of this enzyme is apparent from the precursor feeding studies described above, the general lability and low abundance of plant cytochrome P450 enzymes probably rules out cloning by classical enzyme purification. Therefore, an alternative strategy is to use a polymerase chain reaction (PCR) based method, using RNA isolated from a system in which the enzyme activity is highly induced, namely vanilla cell cultures exposed to an elicitor, such as malic acid. This strategy is facilitated by the recent appearance of many plant P450 sequences in the gene data bases, as this allows the design of primers that can be used for PCR amplification of unknown P450 sequences. According to recent reviews on plant cytochrome P450s, several hundred different sequences have been listed that appear to encode such enzymes. However, for most, the function is not yet known.

The genes encoding cytochrome P450s are highly divergent at the nucleotide sequence level. Nevertheless, these enzymes do contain conserved sequence motifs in their open reading frames sufficient for the design of PCR-based cloning strategies. Specifically, a highly conserved motif (F-G-R-C-G), that includes the cysteine residue which binds the heme group necessary for catalysis by this class of enzyme, is present in all known P450s, and is located near the carboxyl end of the protein. Forward and reverse oligonucleotide primers are constructed for PCR amplification. These are based on sequence motifs surrounding nucleqtides 500, 1050, and 1400 (the heme binding region) of the alfalfa cinnamate 4-hydroxylase cytochrome P450, one of the best characterized plant cytochromes P450. Degenerate primers are constructed. In particular, inclusion of each of the 4 nucleotides (A, T, G, C) at the 3′ end, optimizes the amplification of novel P450 sequences. The PCR primers also contain restriction endonuclease sites at their ends to facilitate cloning of the PCR products.

The template for PCR amplification, as described above, is double-stranded DNA produced by reverse transcription of RNA from vanilla cell culture exposed to an elicitor, such as malic acid. After separation of PCR products by gel electrophoresis, the amplified band containing multiple P450s is cut out from the gel, and cloned into E. coli. The inserts in individual clones are analyzed by gel electrophoresis to determine insert size, followed by restriction enzyme analysis in order to place the clones into classes. Central to our experimental design, we then use the various P450 inserts as labeled probes for northern blot hybridization to RNA isolated from elicited and unelicited cultures. P450s that are present in the elicited but not the unelicited culture are taken to the final stage of the analysis, the functional expression for enzymatic activity.

Initially, the P450s will be expressed in E. coli. For such a method to be successful, it is usual to co-express, in trans, a NADPH cytochrome P450 reductase. This could be from various sources, but the P450 reductase from the bacterium Pseudomonas, or the plant Arabidopsis is used initially. Enzymatic assay in bacteria is facilitated by a staining method for colonies expressing an enzyme capable of forming ortho-dihydroxyphenols, as described by Yabannavar and Zylstra (1995). In an alternative strategy, the DNA is expressed in yeast, using the pYEUra3 expression vector that has been successfully used for yeast expression of the alfalfa cinnamate 4-hydroxylase P-450 (Fahrendorf and Dixon, 1993).

Once cloned and expressed, the cytochrome P450 that catalyzes the hydroxylation p-hydroxybenzyl alcohol to 3,4-dihydroxybenzyl alcohol is further analyzed in order to determine its substrate specificity. For example, is the enzyme promiscuous in its specificity, or does it only hydroxylate p-hydroxybenzyl alcohol?

The 3-hydroxylase cDNA is cloned into a suitable expression vector for vanilla transformation. Initially, the cDNA is expressed constitutively, driven by the rice actin or the maize ubiquitin promoters. These promoters are very effective in monocots. Transformation of vanilla is described in detail in a later Example.

II. Use of Auxotrophs

A major technical problem in the isolation of a particular plant gene is the method of screening cloned libraries containing many thousands of DNA or cDNA sequences for the one desired gene sequence. Recently, the method of functional complementation has been applied to the screening of libraries of cloned eukaryotic cDNA sequences. While this is one of the many technologies of gene isolation, it is particularly appealing in its power and simplicity. In this method, a mutant bacterial strain with a selectable phenotype is transformed with a higher plant cDNA library which carries full-length copies of messenger RNA molecules in the expressible form. The desired cDNA sequences are actually selected in this method, which is more powerful than screening methods in finding very rare sequences in the library. For example, mutations resulting in nutrient auxotrophies in the test bacteria have been readily used to identify the homologous gene from higher plants. The test bacteria can only grow and form colonies if they have received the homologous gene from the library which restores their nutritional deficiency. However, this method is not limited to mutant bacterial strains alone. Indeed, any selectable phenotype can be used for the complementation test and a wide variety of test organisms are possible. The best selectable phenotype is growth of the test cells. In the case of HBA-hydroxylating activity, a test strain may only be able to use HBA as a carbon source if this substance is first hydroxylated. For example, recently, the pathway of HBA metabolism has been studied in the fungus Aspergillus fumigatus (ATCC 28282) as part of the metabolism of p-cresol. The data indicate that HBA metabolism requires the hydroxylation by a monooxygenase enzyme for further metabolism. As this organism grows on p-cresol as a sole source of carbon and energy, the development of a mutant strain suitable for complementation testing for a plant HBA monooxygenase activity is expected to be successful. In this instance, cDNA clones encoding higher plant HBA-hydroxylating enzymes, such as the enzyme involved in the vanillin biosynthetic pathway, are cloned by selecting for microorganisms that are capable of growth on medium containing HBA as the sole carbon source.

### EXAMPLE 8

### Purification and Characterization of Vanillyl Alcohol Dehydrogenase (VAD), Cloning of VAD-Encoding cDNA and Gene, and Regulation of VAD Gene Expression

Application of vanillin to the growing medium of embryo culture results in the rapid uptake and reduction of the applied compound to vanillyl alcohol as illustrated in FIG. 3. Further more, application of 3,4-dihydroxy-benzaldehyde, a vanillin precursor also results in the accumulation of vannilyl alcohol indicating that the tissue has high capacity for the reduction of vanillin to produce vanillyl alcohol. Similarly, vanilla embryo culture that can be elicited by various elicitors to produce vanillin accumulates vanillyl alcohol as a final product. It is important to state at this stage that all the intermediates in the pathway are found mainly as glucosides.

We have purified and characterized the activity of VAD that catalyze the reduction of vanillin to vanillyl alcohol. The enzyme was identified as an NADH or NADPH-dependent alcohol dehydrogenase. The purification protocol was as set forth below.

Crude Enzyme Extract:

Tissue was homogenized in 0.05 M actetate-Na buffer pH 4.0 (in proportion, Ig of the tissue and 5 ml of the buffer) in an ice bath, using Polytrone homogenizer, at 20,000 revolutions of the blade per min. The homogenate was centrifuged at 13000 g for 15 min at 4° C. and the supernatant served as the crude enzyme source.

Ammonium Sulfate Fractionation and Molecular Sieving:

Crude enzyme extract (100 ml) was supplied with ammonium sulfate in an ice bath up to 60% of saturation (44.4 g of Ammonium sulphate per 100 ml). The precipitate was centrifuged and discarded. The supernatant was supplied with ammonium sulfate up to 90% of saturation (total 64.6 g per 100 ml of crude extract). The precipitate was collected (10 min centrifugation at 10,000 g), dissolved in 7 ml of extraction buffer and subjected to molecular sieving at Sephacryl S-300 High Load Column (LKB Pharmacia) 2.6×60 cm, in 0.1 M pH 4.0 acetate-Na buffer. The flow rate was adjusted to 1 ml/min and 5 ml fractions were collected. The protein elution profile was monitored at 280 nm. Five fractions (of a total of 50) containing dehydrogenase activity (130-155 ml of the column eluate) were collected.

Affinity Chromatography on Red Sepharose CL 6B(LKB-Pharmacia):

The active combined fractions from the Sephacryl column (24 ml) were applied on 2.5×3 cm Red Sepharose CL 6B column, equiluibrated with 0.05 M pH 4.0 acetate-Na buffer. The column was developed with 0.5 M Tris/HCL buffer pH 7.4 in 0.0-1.0 M NaCl gradient (total gradient volume=70 ml) and 2.5 ml fractions were collected. Vanillyl alcohol dehydrogenase was released from the column between 0.3-0.4 M NaCl. Fractions containing VAD activity were dialysed overnight against 0.05 M acetate-Na buffer pH 4.0 and concentrated up to 50 times using Minicon concentrating filters (Amicon).

Polyacrylamide Gel Electrophoresis:

Concentrated enzyme extract was used for native and SDS polyacrylamide gel electrophoresis. In native electrophoresis, two active bands (corresponding to protein bands localized with Coomassie brilliant blue) of molecular weight between 43 and 67 kDa were found. SDS electrophoresis revealed 3 still-active protein bands of 20 kDa, 37 kDa and 40 kDa, respectively.

The pH optimum for the enzyme extraction was at 3.0 and optimum activity was obtained at pH 4.0. The subunit molecular weight determined on the basis of electrophoretic mobility in the presence of SDS was around 43 kDa. Table 13 indicates that VAD shows preference toward C₆-C₁phenolic compounds and no activity toward C₆-C₃ phenolics. 3,4-dihydroxy-benzaldehyde and vanillin appear to be the most preferred substrates while affinity to other C₆-C₁ aldehydes or acetaldehyde is lower.

TABLE 13
Substrate specificity of alcohol dehydrogenase
from Vanilla planifolia embryo culture.

| | Activity (nmoles | |
|---|---|---|
| | gfwt⁻¹ · min⁻¹) | |
| Substrate | NADH | NADPH |
| 1. Acetaldehyde | 0.26 | — |
| 2. Benzaldehyde | 0.33 | 0.18 |
| 3. 4-hydroxybenzaldehyde | 0.32 | 0.14 |
| 4. 3,4-dihydroxybenzaldehyde | 1.89 | 2.11 |
| 5. 4-hydroxy-3-methoxybenzaldehyde | 1.26 | 0.97 |
| 6. 4-methoxy-3-hydroxybenzaldehyde | 0.00 | 0.00 |
| 7. 4-hydroxy-3-ethoxybenzaldehyde | 0.42 | — |
| 8. 4-methoxy-3-ethoxybenzaldehyde | 0.00 | — |
| 9. Cinnamylaldehyde | 0.00 | — |
| 10. 4-hydroxy-3-methoxycinnamylaldehyde | 0.00 | — |

Sequence information from the purified VAD protein is used to design primers to clone the gene encoding VAD, and the cloned gene is used for the creation of a VAD antisense gene using established methods (below). Vanilla tissue culture is transformed with the antisense gene and the tissue assessed for an expected attenuation in the activity of VAD and a corresponding reduction in vanillyl alcohol accumulation concomitant with an increase in the level of vanillin.

Sequencing of the VAD Protein:

The purified VAD is purified further to homogeneity, using conventional chromatographic approaches such as chromatofocusing and hydrophobic interaction chromatography. Tryptic peptides from the purified protein are sequenced by automated Edman degradation and used to design oligonucleotide primers for PCR amplification (He and Dixon, Arch. Biochem. Biophys. 336: 121-129, 1996). Since the relative position of the tryptic fragments in the VAD sequence may not be known, degenerate oligonucleotide primers based on regions of minimal degeneracy in the genetic code are designed for each peptide in both forward and reverse orientations, and the various primer combinations evaluated. Oligonucleotide sequences are synthesized as outlined by He and Dixon (1996).

Cloning of the VAD Gene:

Production of cDNA Library. High levels of VAD are produced constitutively in vanilla embryo culture. Embryo culture cells are harvested on nylon mesh, frozen in liquid N2, and stored at −70 C. A cNDA library for DNA probing and expression is constructed from poly(A)+ RNA extracted from vanilla embryo culture cells using the LambdaZAP system.

PCR Screening.

Template DNA for PCR amplification is obtained by boiling a portion of a vanilla cDNA library as previously described (Junghans et al., Plant Mol. Biol. 22: 239-253, 1993). Amplified fragments are cloned, and sequenced to check that they contain sequences corresponding to one or more of the cryptic peptides, along with sequence diagnostic for dehydrogenases. The Lambda ZAP cDNA library from vanilla tissue are autoexcised into p-Bluescript, and screened with PCR fragments that had been 32-labeled by random priming. Positive plaques are identified by autoradiography. Full length clones are sequenced on both strands, and functional identification is performed by expression in E. coli.

Immunoscreening.

In another approach the expression library containing cDNAs derived from transcripts from vanilla embryo culture cells are screened with an antiserum raised against VAD. E. coli (XL-1 Blue cells obtained from Stratagene) are infected with the library, and positive clones selected be purified by several rounds of screening and processed to homogeneity. Sequencing of VAD cDNA clones is according to standard methods.

Regulation of Expression

Expression of VAD in Vanilla Tissues and Cell Cultures.

The expression pattern of VAD in vanilla beans and embryo cultures is determined by northern blot hybridization, and the genomic organization of VAD determined by Southern blot hybridization. RNA is extracted as previously described (Logemann et al., Anal. Biochem. 163: 16-20, 1987), total RNA separated and transferred to and fixed onto cellulose membranes (Jorrin and Dixon, Plant Physiol. 92: 447-455, 1989) and hybridized to an internal coding fragment of VAD. Genomic DNA is isolated from vanilla culture, digested with restriction enzymes, fractionated by electrophoresis and hybridized to a labeled VAD using standard procedures.

Antisense.

Double stranded full length VAD clones are sequenced in both directions and used to construct a VAD antisense gene.

Transformation of Vanilla Cultured Cells with VAD Antisense Constructs.

The VAD cDNA is cloned, in both sense and antisense orientations, into a suitable expression vector for vanilla tissue transformation. The idea is that some sense transformants may exhibit highly reduced VAD activity due to epigenetic co-suppression. Initial transformations focus on achieving constitutive expression driven by the rice actin or the maize ubiquitin promoters. The cDNA clone is introduced by particle bombardment as described in a later Example.

Analysis of Transgenic Plants.

Putative transgenic plants are screened for the VAD antisense transgene by PCR using primers designed to sequences within the selectable marker gene. Transformation is confirmed by Southern border analysis using a VAD cDNA probe. Screening for expression of VAD activity in transformed vanilla culture is done spectrophotometrically based on NADH oxidation as previously described (Biscak et al., Arch. Biochem.Biophys. 215: 605-615, 1982, Longhurst et al., J. Food Biochem. 14: 421-433, 1990) and VAD transcripts determined by northern blot hybridization. In addition, the culture is extracted and analyzed by HPLC for the intermediates in the vanillin biosynthetic pathway (Havkin-Frenkel et al., 1996), in particular the levels of vanillin and vanillyl alcohol. The enzyme (VAD) is extracted as described above.

### EXAMPLE 9

### Transformation of Vanilla planifolia

Cultured Vanilla planifolia was transformed by the procedure set forth below:

1. Vanilla culture was grown on a basic medium agar plates containing 2,4 dichloro-phenoxyacetic acid (2,4-D), was subcultured 3 times during 4 months according to the procedures described above. Cultures were kept at 25° C., 15% humidity, under illumination of 80 λE/sec/m².

2. Soft callus from the culture was chopped to very small pieces.

3. The pieces were washed with regular (basic) liquid media (G medium as described above) and transferred to agar plates containing basic media plus 1% polyvinylpyrrolidone (PVP) and to plates containing basic media plus 1% charcoal.

4. The green pieces from each plate were washed every day for 5 days and transferred to new PVP/charcoal plates.

5. Only the green pieces were collected and placed on a disk in a basic medium plus 0.6 M mannitol agar plates for 4 hours.

6. Particle bombardment was done according to standard methods, using ACT 1 D plasmid (McElroy et al., Plant Cell 2: 163-171, 1990) having the rice actin promoter fused to a beta-glucoronidase “GUS” coding sequence. The gun used was a Biored PDS 1000/He Biolistic Delivery System, used at 11,000 psi at a distance of about 10 cm. The samples were kept over night after bombardment.

7. The samples were transfered to a basic media agar plates for 24 hours.

8. The tissue samples were stained for “GUS” activity using X/Gluc, and incubated at 37° C. over night.

Results of GUS staining demonstrated that the vanilla tissue had taken up the plasmid and were able to express GUS. This indicates a successful transformation of vanilla callus tissue using the above-described procedure.

### EXAMPLE 10

### Activity Analysis of Cloned V. planifolia p-Coumaric Acid Chain-Shortening Enzyme

Utilizing the partially purified p-coumaric acid chain shortening enzyme (CSE) described in Example 6 and other methods, the inventors obtained a nucleic acid molecule that encodes the enzyme. The nucleotide sequence of this molecule is set forth herein as SEQ ID NO:1, and the sequence of its encoded protein is set forth as SEQ ID NO:2.

The isolated nucleic acid molecule was inserted into appropriate expression vectors and used to transform bacterial or yeast cells. Transformants expressing the CSE were selected by their ability to take up coumaric acid from the culture medium. These cellular expression systems were utilized in activity assays for the enzyme as follows.

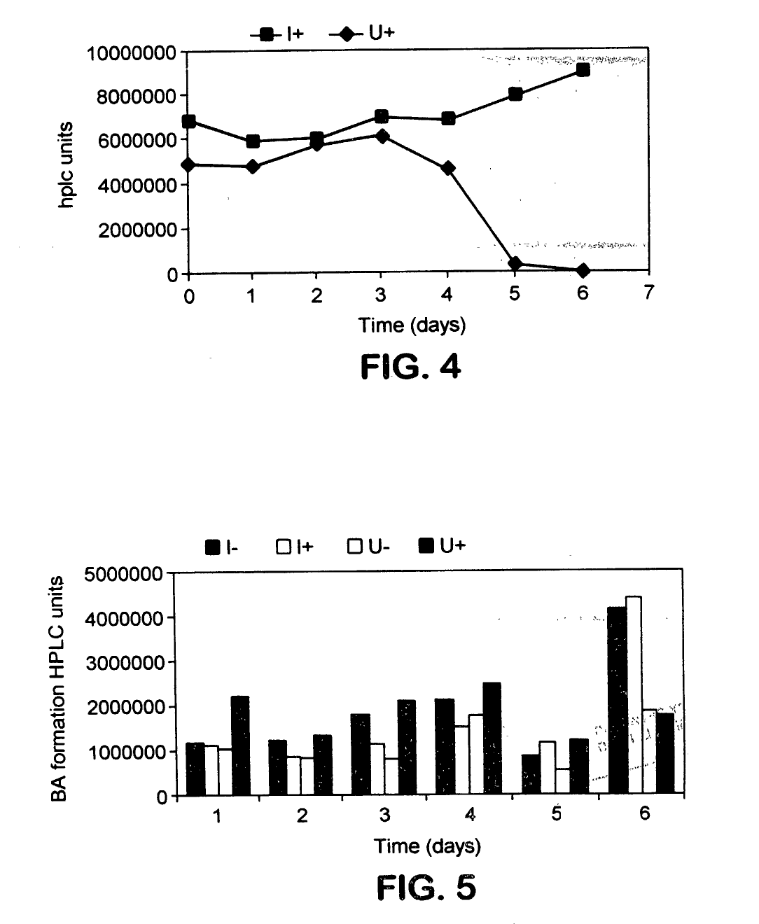

Coumaric acid uptake from the medium was used as one basis to measure activity. A selected transformed cell was grown in culture. Coumaric acid was added to the media and its disappearance therefrom was measured daily for six days. Two parallel cultures were grown, one in which enzymatic activity was induced by the addition of methanol at Day 3, the other which was uninduced. Results of a typical experiment are shown in FIG. 4. As can be seen, the induced culture was depleted of coumaric acid subsequent to the induction, indicating uptake of the coumaric acid by the cells, presumably as a result of CSE activity.

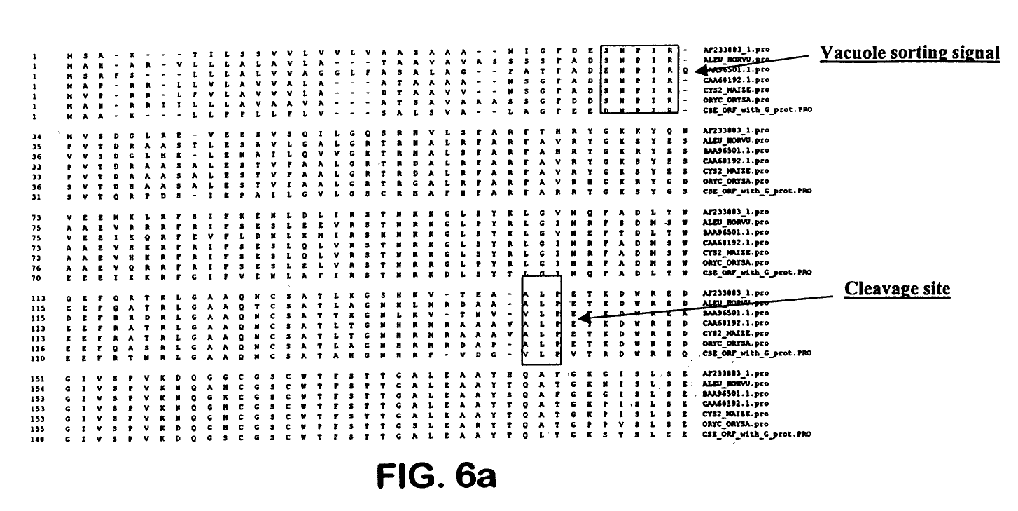

Next, CSE activity was measured in recombinant cells based on formation of the enzymatic product, p-hydroxybenzaldahyde (BA). Results are shown in FIG. 5. It should be noted that these results do not subtract background from the measurements. As can be seen, the recombinant cells were capable of forming p-hydroxybenzaldehyde whether or not coumaric acid was fed in the medium. Further, cells induced with methanol had significantly greater activity than did un-induced cells.

### EXAMPLE 11

### Agrobacterium-Mediated Transformation of Vanilla planifolia Embryo Culture

Vanilla embryo culture was grown in B-5 solid (0.8% agar) media (in some cases supplemented with μM amounts of BA (here, benzyladenine) and pM amounts of NAA (napthaleneacetic acid)) for 6-12 months with sub-culturing every 2 weeks in the dark. The culture became yellowish and softer and undifferentiated. This culture was used for genetic transformation and called “Red”. The Red culture was transferred to 2 mg/L Zeatin for further changing to softer callus. This is referred to as the “Z culture”. Other embryo cultures were grown on a raft in the light for 3 years. These also were used for transformation and called EMR. The EMR was also cultured every two weeks on B-5 media. For Agrobacterium transformation, we used three different cultures: (1) EM (Embryo) growing on agar or liquid media B-5 without plant hormones; (2) the EM used for transformation was taken from a liquid culture, Red (10 μM BA/5 mM NAA); and (3) Z (10 μM BA and 5 μM NAA, followed by 2 mg/L Zeatin.

Frozen Agrobacterium PMJ805 were streaked on LB agar plate containing 5 mg/L tetracycline. The plate was incubated at 30° C. for 2 days. Next, one single colony was transferred to LB liquid (100 ml in 500 ml flask) containing 5 mg/L tetracycline for 2 days on a rotary shaker at 30° C. Next, 0.1 ml of the culture was transferred to a fresh LB liquid media that contained 0.1 mM tetracycline and 0.1 mM acetophenone. Twenty-four hours later the culture was centrifuged at 5000 g for 15 minutes. The precipitate was resuspended in B-5 liquid media. This preparation was used for co-cultivation with the vanilla culture.

The Red, EM and Z culture were cut to 1-3 mm in diameter and bombarded once with gold a day before the co-cultivation. The EMR were cut in small pieces in such a way that each piece has a little shoot. The bacteria preparation was mixed with the tissue and infiltrated twice. Then the following were applied:

1. After 15 minutes, the culture pieces were transferred to B-5 supplemented with acetophenone 0.1 mM for 3 to 6 days.

2. The liquid containing bacteria in B-5, plus the culture, was left overnight and then transferred to B-5 agar media supplemented with acetephenone at 0.1 mM. Then the culture was left for co-cultivation for 3-6 days to kill the Agrobacterium.

3. After the period of co-cultivation, the cultures were transferred to B-5 and antibiotics (to kill the Agrobacterium) for 1 week, and then transferred to a media containing 4-5 mg/l bialaphos or a similar suitable agent We found that 4 mg/l was a good concentration for selection Red and Z, and 5 mg/l for EM and EMR cultures. Controls with no co-cultivation (gold bombardment only) were included.

The Agrobacterium cells were killed in the following way. 500 mg Augmentin/l in B-5 in liquid for 3-6 days, transferring to fresh media as needed, then placed on agar media, plus bialaphos, or directly on agar plate with 500 mg Augmantine for 3-6 days. Other antibiotics were applied, such as cefatoxin 500 mg/l or combination of Cefatoxin and Vanomycine. In case of a persistent presence of Agrobacterium, PPM were applied. The culture was dipped in full-strength PPM and transferred to B5 and 0.5 ml/l of PPM. In other cases, the culture were dipped or infiltrated in 1 g/10 ml. Cefatoxin for a few seconds.

In recalcitrant cases, when the Agrobacterium were still growing around the tissue, the pieces of callus were washed with sterilized water with Tween to reduce the sticking of bacteria to the culture and then washed 3 times with clean water and dried and then the antibiotics were applied. Excess of antibiotics were dried on a fresh paper towel.

The above procedure was continued during selection. When the tissues appeared free of Agrobacterium, the antibiotics were reduced or eliminated. The combination of high concentration of antibiotics and bialaphos reduced the growth and affected the incorporation of the DNA.

Another way of selection that prevents the culture from slowing growth is to place the culture first on 2-3 mg./l biolaphose for 2-3 weeks and then transfer to 4-5 mg/l bialaphos. The culture suffers from the combination of bombardment/antibiotics/bialaphos and it was difficult to obtain rapid growth. The better approach at that time was when 80% of the culture was dead and all the controls were dead within 1-2 months of co-cultivation. The remaining 20% was transferred back to regular B-5 (agar and liquid) for growing and then placed back on bialaphos. After 6-8 months on selection media, culture was transferred back to B-5.

The next approach was:

1. Waiting for selection for 1-4 months after co-cultivation. The culture suffers from cutting/bombardment/antibiotics. After this period, when new growth appears steadily, we started the selection.

2. Selecting for 1-2 months, then back to B-5 after 1-2 months, or when the culture tripled its size, back in selection.

The DNA was extracted using the Qiagen kit and used for PCR with primer for the bar gene. The control was non-transformed culture.

### EXAMPLE 12

### Further Purification and Characterization of 4-Hydroxybenzaldehyde Synthase from Vanilla planifolia Green Embryo Culture

Purification and Characterization

Crude extracts of V. planifolia cell cultures demonstrated 4HBS activity when incubated with 4-coumaric acid and dithiothreitol (DTT) at pH 8.0. HPLC analysis indicated time-dependent production of a new compound, identified as 4-hydroxybenzaldehyde by comparison of its chromatographic mobility, UV absorption spectrum and mass spectrum with those of an authentic standard. Conversion of 4-coumaric acid to 4-hydroxybenzaldehyde occurred spontaneously at a low rate under the assay conditions in the presence of DTT. This conversion was subtracted from the enzyme-catalyzed rate for calculations of activity.

4HBS was purified from crude cell culture extracts by a four-step sequence of ammonium sulfate fractionation, hydrophobic interaction chromatography, ion exchange chromatography and size exclusion chromatography (see Methods). Fractionation of the 40% ammonium sulfate supernatant on Phenyl-Sepharose Cl 6B resulted in the bulk of the 4HBS activity eluting in a broad double peak at around 1.0-0.8 M ammonium sulfate. When re-fractionated on an FPLC MonoQ ion exchange column, approximately 50% of the 4HBS activity remained unbound and eluted in the initial wash, with the majority of the remaining activity eluting as a biphasic peak soon after the start of the NaCl gradient. The unbound material (4HBSI) was further fractionated by gel filtration on Superdex 2000 HR. The bulk of the activity (IB) appeared as a rather broad peak that eluted between the marker proteins egg albumin (Mᵣ 44,000) and equine myoglobin (Mᵣ 17, 000), with a smaller peak (IA) of higher Mᵣ. An almost identical elution pattern was observed when the 4HBS activity that was retained on MonoQ (4HBSII) was separated on Superdex 200 HR, suggesting the two forms of 4HBS resolved on ion exchange chromatography may be closely related. That these forms are inter-convertible is shown by the fact that re-chromatography of fraction IIB on Mono Q resulted in an almost identical activity profile to that seen originally, with major peaks at the elution volumes of the original fractions I and II. However, re-chromatography of fraction I resulted in a single peak that was not retained by the column.

The molecular weights corresponding to the peak 4HBS activities in fractions IIA and IIB were determined by comparison of the peak elution volumes to those of standard molecular weight markers run through the same Superdex 200 HR column. From this analysis, the holoenzyme molecular weights corresponding to the peaks of IIA and IIB were 79.4 kDa and 28 kDa, respectively. The width of peak IIB was 2.5-3 times greater than that of the molecular weight standards with which the column was calibrated, suggesting heterogeneity of activity within this fraction.

The major 4HBS activity peaks were further analyzed by SDS-PAGE. First, the V. planifolia cell extract was resolved by ammonium sulfate fractionation, hydrophobic interaction chromatography and gel-filtration on Superdex 200 HR. Peak B was then further resolved by chromatography on Mono Q, and fractions analyzed for 4HBS activity and constituent proteins. Extracts were fractionated by hydrophobic interaction chromatography and ion-exchange on MonoQ. Peak I from the MonoQ was re-fractionated on MonoQ (smaller fraction size, same elution profile), and then further fractionated on the Superdex 200 HR. The second peak was collected and termed 4HBSA. Peak II from the MonoQ fractionation of the same extract was further fractionated on the Superdex 200 HR and fractions 16-19 were collected. This fraction was termed 4HBSB. All fractions with high 4HBS activity contained a protein of Mᵣ 28 kDa and several proteins of between 31 and 45 kDa.

The chain shortening enzyme activity was quite specific for 4-coumaric acid. Cinnamic, caffeic, sinapic and o-coumaric acids were not substrates, and low activity (approximately 2% of the activity with 4-coumaric acid) was obtained with ferulic acid, which was converted to 3-methoxy-4-hydroxybenzaldehyde. These results are consistent with earlier proposals for the vanillin biosynthetic pathway in beans of V. planifolia, in which coumaric acid may be the major precursor and ferulic acid aminor precursor (reviewed in Dignum et al., 2001).

The pH optimum for the reaction was 8.0. Chain shortening of 4-coumarate required the presence of a thiol reagent. Dithiothreitol (DTT), dithioerythritol, and coenzyme A were all equally effective, whereas 2-mercaptoethanol, glutathione, and cysteine were much less effective (Table 1). At 10 mM 4-coumarate, the reaction rate increased linearly with concentrations of DTT up to 20 mM. The reaction with 4-coumarate and DTT was almost completely inhibited in the presence of 1 mM ascorbate (Table 1). The thiol-dependent 4HBS activity remained relatively constant during growth of the cultures, with an average production of 4-hydroxybenzaldehyde of around 88 mmol min⁻¹ mg protein⁻¹ over a 40 day period.

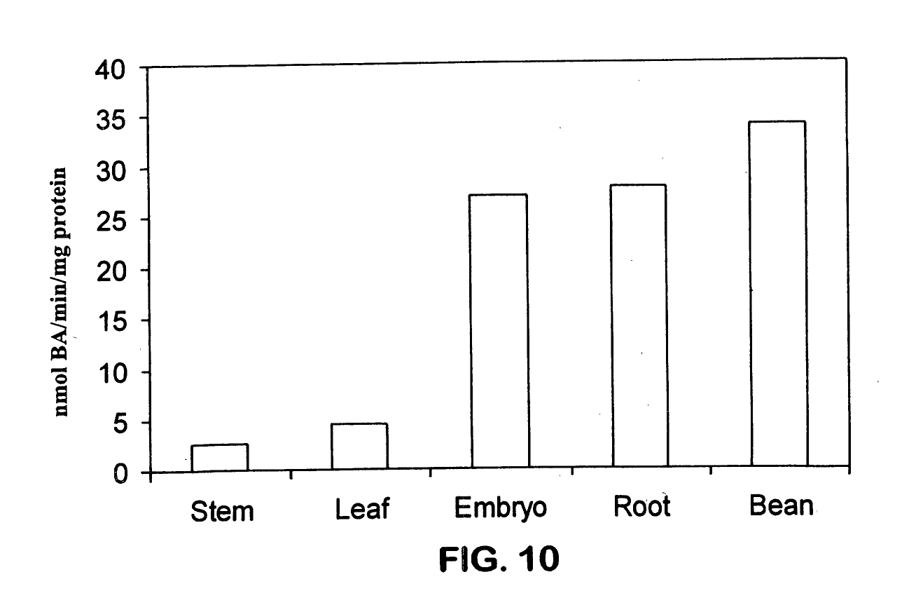

4HBS activity was observed in stems, roots, leaves, pods and embryo cultures of V. planifolia, with highest specific activity in roots (FIG. 10). The effect of developmental age of the pods on 4HBS activity was also determined. Maximum activity occurred months post-pollination; vanillin appeared in the pods between 4 and 5 months post-pollination, with maximum levels after 6 months. The activity could not be induced further in the cell cultures by treatment with yeast elicitor or transfer to media containing various concentrations of gibberellic or abscisic acids.

Kinetic studies with 4HBS purified through the ammonium sulfate and hydrophobic interaction chromatography steps revealed the unusual finding that the reaction rate did not saturate with concentrations of 4-coumaric acid up to as high as 100 mM, and therefore a Km value could not be calculated. This phenomenon has been observed previously for 4HBS activity from L. erythrorhizon (Yazaki et al., 1991). Such kinetics suggest some degree of positive cooperativity, consistent with a multimeric enzyme system (Ricard et al., 1974).

The oxidative pathway proposed for chain shortening of cinnamic acids involves generation of a coenzyme A thioester. To determine whether this type of activity was present in the V. planifolia cultures, crude extracts and purified enzyme were incubated with ATP, Mg⁺⁺ and coenzyme A (Table 1). Significant 4-hydroxybenzaldehyde production occurred when 4-coumarate was incubated with 10 mM CoA and 10 mM MgATP, but it was nevertheless less than production with CoA alone, suggesting that the reaction did not occur through production of coumaroyl CoA. No 4-hydroxybenzoic acid, the product of oxidative chain shortening, was detected. Furthermore, incubation of crude V. planifolia extracts with 4-coumaroyl coenzyme A did not result in production of 4-hydroxybenzoic acid or 4-hydroxybenzaldehyde. Thus non-oxidative chain shortening appears to be the major route to vanillin precursors in V. planifolia cell cultures.

### Discussion

Previous studies on the enzymes leading to 4-hydroxybenzoate derivatives in plants led to proposals for at least three different mechanisms for the chain-shortening reaction (Funk and Brodelius, 1990; Yazaki et al., 1991; Löscher and Heide, 1994). The present data provide a more detailed biochemical characterization of a plant chain-shortening enzyme system, and confirm a non-oxidative mechanism most likely involving a hydrolyase activity that proceeds by hydration of the side chain 2,3 double bond of 4-coumaric acid with subsequent cleavage of the side chain to yield acetate and 4-hydroxybenzaldehyde. This reaction, which has been previously proposed as the first step in formation of 4-hydroxybenzoic acid in L. erythrorhizon and carrot cell cultures (Yazaki et al., 1991; Schnitzler et al., 1992), is presumed to involve the unstable 4-hydroxyphenyl-β-hydroxypropionic acid (FIG. 1) as an intermediate, although this compound was not detected in the present work, or in previous studies.

The exact relationship between the different forms of V. planifolia 4HBS resolved in the present study is not clear. The possibility that 4HBS activity comprises a complex of several different protein subunits is suggested by the unusual kinetics of the reaction, the breadths of the peaks of highly purified enzyme obtained by gel filtration, and the apparent interconvertibility of the major forms of the activity when considered in relation to their similar polypeptide compositions.

One study on 4-hydroxybenzoate synthesis in L. erythrorhizon provided convincing evidence for a pathway involving oxidation and cleavage of 4-coumaroyl CoA to 4-hydroxybenzoyl CoA and acetyl CoA in a thioclastic reaction with requirement for NAD (L\ddot{o}scher and Heide, 1994). In this study, non-oxidative formation of 4-hydroxybenzaldehyde was also observed, although at much reduced rates compared to the oxidative conversion. It was concluded that the non-oxidative pathway could represent either an alternative route to 4-hydroxybenzoic acid, or could be an artifact of the assay (Löscher and Heide, 1994). Our data do not formally rule out this latter possibility, because it is conceivable that the enzymatic reactions we are measuring are relatively non-specific, and the nature of the reductant used for the 4HBS reaction in planta still remains to be determined; it is clearly not DTT, and the physiologically occurring reductant glutathione was much less effective than DTT. It is possible from our data that CoA could be used in vivo, but in a reaction that did not involve formation of the CoA ester. Whatever the in vivo mechanism, the apparent absence of the alternative oxidative pathway in the vanilla tissues points to a biological function for the non-oxidative pathway. The biological role of the non-oxidative 4HBS in vanillin biosynthesis will best be directly determined by genetic or reverse genetic approaches.

### Materials and Methods

Plant Materials

To initiate the embryo cultures, green V. planifolia beans (4-5 months old) from Indonesia were sterilized, placed in petri dishes, and transferred to fresh solid media every two weeks. The seeds embedded in the beans germinated after 3 to 6 months. The embryo cultures, which contain differentiated cell aggregates, were maintained in media containing Gamborg's (Gamborg et al., 1968) B-5 salts, and 20 g/l sucrose. Vitamins and microelements were added to a final concentration of (mg/l): L-glycine (2.0), myo-inositol (50), thiamine-HCl (2.0), nicotinic acid (0.5), D-biotin (0.25), pyridoxine-HCl (0.25), boric acid (1.5), zinc sulfate (1.5), cupric sulfate (0.05). To prevent microbial contamination, 42 mg/l Cefotaxime sodium and 33 mg/l Vancomycin-HCl were added to the media. Solid media also contained 0.8% agar. Liquid cultures were grown in the light at 50 _E sec⁻¹ m⁻² at 25° C. on a gyrotary shaker at 180 rpm.

Cultures grown in liquid media were subcultured at approximately two week intervals by sieving the cultures, cutting each aggregate into several pieces, and transferring 4 g to 5 g of fresh cells to 100 ml of new media. Cultures grown in petri dishes were subcultured every four weeks (Havkin-Frenkel et al 1996).

The cell fresh weight was measured after sieving the cultures, rinsing with deionized water, and drying with a paper towel. The cell dry weight was determined by drying approximately 1 g of fresh cells in a laboratory oven at 65° C.

Extraction of Phenolic Compounds:

The procedures used to extract phenolics from the cells and to analyze the extracts by HPLC are based on the methods developed by Havkin-Frenkel et al. (1996). First, 10 ml of 50 mM sodium acetate, pH 5.0, was added to 3 g of fresh cells. The samples were placed in boiling water for 3 min and then homogenized (IKA-Labortechnik Ultra-Turrax T25, Staufen, Germany). After adding almond _-glucosidase (Sigma, G-0395, St. Louis, Mo.) to a final concentration of 0.2% (w/v), the mixture was incubated at 37° C. for 5 hr. Twenty five ml of 95% ethanol was then added and the samples kept at 37° C. overnight. The extract was filtered using Fisherbrand P8 filter paper (Fisher Scientific, Pittsburgh, Pa.) and the ethanol removed by rotary evaporation. The filtrate was extracted twice with ethyl acetate, the pH adjusted to 3.0 with HCl, and the aqueous fraction extracted a further two times, with the four ethyl acetate fractions then being combined. The organic fractions were dried through anhydrous magnesium sulfate, evaporated on a heat block at 50° C. under nitrogen, and the residue reconstituted with 3 ml methanol containing 1.25% glacial acetic acid and filtered using a 0.45 μm syringe filter (Osmonics, Minnetonka, Minn.).

Assay of 4-hydroxybenzaldehyde Synthase (4HBS):

Crude enzyme extracts were prepared by homogenization of V. planifolia cell culture material in 100 mM HEPES buffer, pH 8.0, containing 10 mM DTT, at 4° C. Low molecular weight compounds were removed by filtration through Sephadex G-25 columns equilibrated in 100 mM Tris-HCl, pH 7.0, containing 10 mM DTT.

Enzyme (10-20 μl) was routinely incubated in a total volume of 200 μl with 100 mM Tris-HCl, pH 8.0, 16 mM 4-coumaric acid and 10 mM DTT at 37° C. for 5-25 min. Reactions were stopped by addition of 200 μl of ice cold 10% acetic acid in methanol, and the mixtures centrifuged at 10,000× g for 10 min. The supernatants were analyzed for production of 4C°hydroxybenzaldehyde by HPLC. For studies on requirements for co-factors and thiol reagents, and substrate specificity, the concentration of 4-coumarate in the assay was 10 mM, total volume 500 μl, and incubation time 30 min.

HPLC and GC/MS Analysis

Production of 4-hydroxybenzaldehyde in routine assays used for enzyme purification was determined by HPLC (Agilent HP1100 HPLC with a G1315A diode array detector and G1311A quaternary pump), monitoring at 280 nm, using a C18 reverse phase column (Waters Spherisorb 5 ODS2 250×4.6 mm), a flow rate of 1 ml/min, and the following gradient: 13% B isocratic for 5 min, followed by an 8 min gradient from 13% B to 20% B, where B Acetonitrile (J. T. Baker, Baker analysed HPLC solvent) and A=1% phosphoric acid in Milli Q water (Millipore Corp.). Vanilla culture crude extracts were analyzed on the same instrument, using the same solvents and flow rate, but with the following gradient: 5% B isocratic for 5 min, followed by a gradient to 10% B in 5 min, then to 20% B in 20 min, and finally to 60% B in 15 min.

The peak designated as 4-hydroxybenzaldehyde was further analyzed, without derivatization, by GC/MS using a HP 5890GC/5971MS system. The column and run conditions were HP-Wax (30 m, 0.25 mm ID, 0.25 μm phase ratio), inlet temp 250° C., 1 ml injection, temperature gradient 1 min at 120° C., 15° C. per min to 200° C., 10° C. per min to 245° C., 25° C. per min to 120° C., at a constant flow at 8 psi.

For analysis of 4HBS substrate and co-factor specificity, HPLC was performed on a Lichrospher 100 column (5 μm reversed phase C-18, 250×4 mm, Merck) with flow rate of 1.5 ml min⁻¹ and UV detection. HPLC conditions were isocratic, with the following solvents: 10% acetonitrile, 25 mM NaAc, pH 3.0 (4-coumaric, ferulic, sinapic and cinnamic acids as substrates); 20% acetonitrile, 25 mM NaAc, pH 3.0 (2-coumaric acid acid as substrate); 10% MeOH in acidified water (caffeic acid as substrate).

Purification of 4HBS:

All procedures were carried out at 4° C. The enzyme was extracted from the tissue with 0.1 M HEPES buffer pH 8.0 containing 10 mM DTT. Ammonium sulfate was added to the crude enzyme preparation to 40% saturation, the resulting precipitate was discarded, and the supernatant fractionated by FPLC on Phenyl-Sepharose Cl 6B. The column was equilibrated in 10 mM Tris-HCl pH 7.0 containing 10 mM DTT (buffer A) with 2M ammonium sulfate. Crude extract in 40% saturated ammonium sulfate was applied to the column, which was then washed with buffer A containing 2 M ammonium sulfate until all unbound proteins were eluted. 4HBS activity was eluted in 0.8 M ammonium sulphate in buffer A. Fractions containing 4HBS activity were pooled, concentrated and desalted using Amicon membrane concentrators with a cut off range of 10,000 Da.

The desalted 4HBS fraction was applied to a MonoQ ion exchange FPLC column equilibrated with 50 mM Tris-HCl pH 7.5 containing 10 mM DTT (buffer B). The 4HBS activity was eluted using a linear gradient of 0-1M NaCl in buffer B and 1 ml fractions were collected. In a separate experiment in which the 4HBS was run a second time through MonoQ, a 0-0.25 M NaCl gradient at pH 8.0 was used, and 0.25 ml samples were collected. Fractions containing 4HBS activity were pooled, concentrated and desalted as described above.

The 4HBS fraction from MonoQ was further fractionated on a Superdex HR 200 FPLC column equilibrated with 50 mM NaPi buffer pH 7.0 containing 150 mM NaCl and 5 mM DTT. Fractions of 0.6 ml were collected and analyzed for 4HBS activity. The column was calibrated with a range of protein molecular weight standards. Fractions containing 4HBS activity were concentrated and freed of NaCl using Amicon membrane concentrators, and stored frozen at −70° C. in 10 mM Tris-HCl pH 7.0 containing 10 mM DTT.

Fractions containing 4HBS activity from MonoQ and Superdex HR 200 were analyzed on 8%-16% SDS-PAGE gels run in Tris-glycine and stained with either Coomassie blue or silver reagent. Table 1. Effects of thiol reagents and co-factors on 4HBS activity in crude extracts from V. planifolia cell cultures. The standard assay contained 10 mM 4-coumaric acid and 26 μg protein in 0.1 M Tris-HCl, pH 8.0, total volume 0.5 ml. Results are the mean and standard deviation from 3-5 replicate assays.

| Additions to assay | Relative 4HBS activity (%) |
|---|---|
| Dithiothreitol (10 mM) | 100 ± 4.7 |
| Dithioerythritol (10 mM) | 101.1 ± 2.2 |
| Glutathione (reduced) (10 mM) | 8.2 ± 0.1 |
| Glutathione (oxidized) (10 mM) | 0.0 ± 0.0 |
| Cysteine (10 mM) | 18.8 ± 0.1 |
| Coenzyme A (1 mM) | 31.0 ± 4.2 |
| Coenzyme A (10 mM) | 100.8 ± 13.2 |
| Dithiothreitol (10 mM) + ascorbate (1 mM) | 1.0 ± 1.0 |
| Dithiothreitol (10 mM) + MgATP (1 mM) | 148.9 ± 10.6 |
| Dithiothreitol (10 mM) + MgATP (10 mM) | 214.2 ± 25.1 |
| Coenzyme A (1 mM) + MgATP (10 mM) | 23.1 ± 1.3 |
| Coenzyme A (10 mM) + MgATP (10 mM) | 85.9 ± 5.6 |

### EXAMPLE 13

### Sequencing, Cloning and Expression of the 4-Hydroxybenzaldehyde Synthase from Vanilla planifolia

### Cloning of a V. planifolia cDNA Encoding the 4HBS Activity

A comparative analysis of fractions from the final gel filtration step of the purification by SDS-PAGE and 4HBS activity assay indicated that the 28 kDa band was the only band for which chromatographic behaviour correlated with 4HBS activity (see Example 12). This band was therefore subjected to tryptic digestion and peptide sequence analysis as described below.

### Isolation and Sequence Analysis of 4HBS cDNA Clones

Four tryptic peptides from the putative 4HBS were sequenced to provide needed information to develop a cloning strategy. Surprisingly, all 4 peptides showed strong amino acid sequence identity to cysteine protease enzymes. Degenerate oligonucleotides were made corresponding to 3 of the 4 peptides (see Methods below), and these were used in PCR reactions in various combinations to amplify putative 4HBS sequences from a V. planifolia embryo culture cDNA library constructed in the ZAP express XR vector (Stratagene, La Jolla Calif.).

The combination of oligonucleotides 1a and 2b resulted in the amplification of an 800 bp fragment that was subsequently used to re-probe the library for isolation of full-length putative 4HBS clones. Twenty positive clones were isolated and 5 of them were completely sequenced. Sequence analysis indicated that they all corresponded to the same gene.

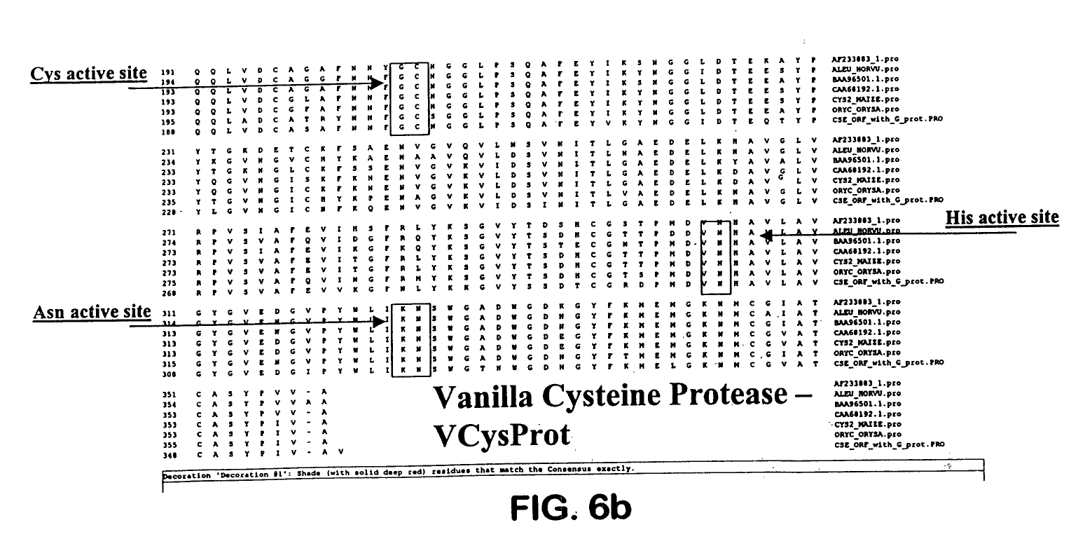

The open reading frame of the putative 4HBS contains 356 amino acids. All 4 peptides obtained from the purified 4HBS enzyme were found within the C-terminal amino acid sequence of the protein. Computer analysis predicted an N- terminal signal peptide, a putative vacuolar sorting signal (NPIR) at amino acid 27, and a prepropeptide with predicted cleavage immediately upstream of amino acid position 136 (FIG. 6, Sequence Alignment). The preproprotein contained the ERFNIN motif characteristic of cathepsin-like cysteine proteases. Potential glycosylation sites were identified at positions ¹²² (NCS) and ²⁵¹ (NIT).

The deduced amino acid sequence of the protein exhibited between 68-73% overall amino acid sequence identity to a number of cysteine protease enzymes from both monocots and dicots, with even greater identity in the mature protein portion of the sequence. The highest amino acid sequence identity of the mature putative 4HBS was to the mature regions of cysteine proteases induced during senescence (Zea mays See 1, 73%, (Griffiths et al, 1997)) and seed germination (Zea mays CCP2, 73% (Domoto et al., 1995)), and to the rice Oryzain (Oryzain gamma, 68% (Watanabe et al., 1991)). The putative 4HBS exhibited 70% amino acid sequence identity to the structurally characterized tobacco cysteine protease (Ueda et al., 2000), 68% identity to Arabidopsis thaliana AtALEU (Ahmed et al., 2000), 69% identity to the Hordeum vulgare thiol protease aleurein (Rogers et al., 1985), 56% identity to the porcine thiol protease cathepsin H (Guncar et al., 1998), 53% identity to rat and human cathepsin H (Ishidoh et al., 1987; Strausberg, 2000) and 39.8% identity to papain (Drenth et al, 1968).

The active sites of cysteine proteases are characterized by a catalytic dyad of a cysteine and a histidine residue, along with a catalytically important downstream asparagine or aspartic acid, embedded in highly conserved regions. These features are fully conserved in the V. planifolia 4HBS, with the presumed catalytic residues, ¹⁶²Cys, ³⁰²His, and ³²²Asn, present within regions of extensive sequence identity as compared to corresponding regions of functionally identified cysteine proteases (FIG. 6).

### Expression and Characterization

Expression of 4HBS in E. coli:

Constructs containing the complete 4HBS open reading frame, or the portion corresponding to the mature protein alone (4HBS-M), were transferred to the vector pET15b for expression of his-tagged protein in E. coli. No protein expression was obtained with the full-length constructs in a variety of different E. coli strains: BL21(DE3), BL21(DE3) RP, BL21(DE3) RIL, BL21(DE3) pLys S or BL21(DE3) pLysE. In contrast, high levels of expression of the his-tagged 28 kDa mature protein could be obtained, although all the protein accumulated in inclusion bodies. Solubilization and renaturation of the 4HBS-M protein according to standard protocols failed to yield an active enzyme with protease or 4HBS activity, possibly because the propeptide may be required for proper enzyme refolding. The expressed inactive 4HBS-M protein was, however, used to generate polyclonal antibodies in rabbits for further studies on 4HBS function, expression and localization.

Functional Characterization of 4HBS in Yeast:

The full-length putative 4HBS open reading frame was cloned into the expression vector pPIC9 for expression in the yeast Pichia pastoris. Constructs harbouring an β-albumin gene, or an empty vector, were transferred to P. pastoris as controls. Induction of transformed cultures with methanol resulted in appearance of the 28 kDa mature protein as detected by gel blot analysis of protein extracts from yeast clones harboring the putative 4HBS open reading frame, indicating that the full length protein is correctly processed in yeast. This protein was not detected in control yeast harboring either the albumin gene construct or empty vector. When 4-coumaric acid was fed to induced cultures of yeast expressing the 4HBS gene, it was removed from the culture medium more rapidly than by induced cultures expressing albumin or harboring empty vector, or by uninduced cultures harboring the 4HBS construct. No 4-hydroxybenzaldehyde was detected in the medium or in cells from induced lines harboring the 4HBS gene, however, this compound is rapidly metabolized by yeast to unknown products. Assays for 4HBS activity confirmed in vitro conversion of 4-coumarate to 4-hydroxybenzaldehyde in crude protein extracts from induced yeast containing the 4HBS construct, but not in extracts from uninduced cultures or induced control cultures expressing albumin. These results indicate that the mature 28 kDa cysteine protease-like protein does indeed encode a 4HBS enzyme. Immunotitration and immunoprecipitation of 4HBS activity:

To further confirm the functional identification of the V. planifolia 4HBS, crude protein extracts from embryo cultures were treated with various concentration of anti-(4HBS) serum or pre-immune serum, and then assayed for 4-HBS activity either directly or after immunoprecipitation with protein A Sepharose. Protein extracts for immunoprecipitation experiments were from cell batches that had been fed with 35S-methionine in order to label the proteins to help confirm specific immunoprecipitation of 28 kDa mature 4HBS subunits. The results in FIG. 6a show that anti-(4HBS) serum inhibited 4HBS activity in crude extracts in a dose-dependent manner. The peak of 4-HBS activity was co-incident with immunodetectable 28 kDa protein, consistent with the antiserum inhibiting 4-HBS activity in the crude extract. Surprisingly, there was also inhibition of 4-HBS activity by pre-immune serum, although to a significantly lesser extent.

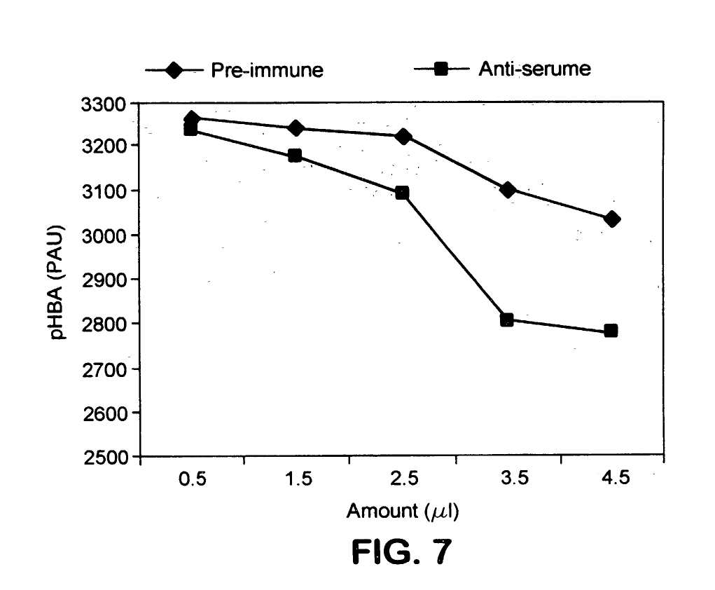

Residual 4-HBS activity in the supernatants of crude extracts treated with anti-(4HBS) or pre-immune sera and immunoprecipitation with Protein A-Sepharose was similar to that seen in antibody inhibition studies, and again there was smaller but significant inhibition by pre-immune serum. Analysis of the immunoprecipitates by SDS-PAGE and fluorography revealed antibody dose-dependent appearance of only a 28 kDa protein precipitated by anti-(4HBS) serum (FIG. 7), and a small but significant appearance of labelled 28 kDa protein in immunoprecipitates from pre-immune incubations. These data link the loss of 4-HBS activity in response to immune and pre-immune sera to precipitation of the 28 kDa protein, and provide confirmatory functional identification of this protein as 4HBS.

V. planifolia 4HBS does not Exhibit Protease Activity

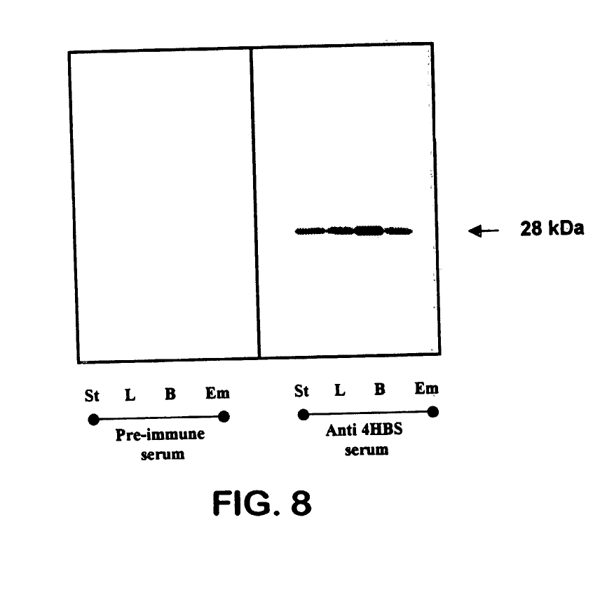

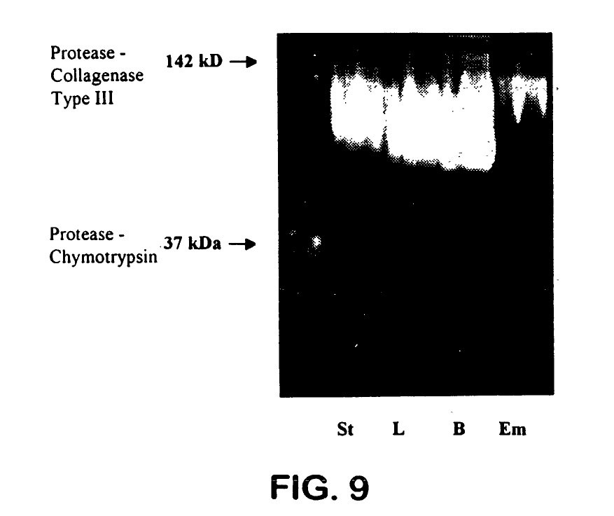

Protein gel blot analysis of crude extracts of V. planifolia stem, leaf, bean and embryo culture revealed the presence of a single band of 28 kDa that reacted with anti-(4HBS) serum (FIG. 8). Despite the strong sequence homology with a number cysteine proteases, there was no protease activity co-incident with the 28 kDa protein apparent in parallel in-gel protease assays using gelatin as substrate (FIG. 9). Similarly, yeast expressing the 4HBS activity exhibited several bands of protease activity, which increased in intensity on induction of the cultures with methanol, although none corresponded to the expressed 28 kDa 4HBS visualized on protein gel blots. Fractions from the MonoQ stage of purification of 4HBS from V. planifolia embryo culture were assayed in parallel for 4HBS activity and protease activity using p-coumaric acid and gelatin as substrates, respectively. The fractions with 4HBS activity contained no detectable protease activity.

Crude extracts and purified 4HBS preparations were assayed in the presence of the cysteine protease inhibitors cystatin (cat. # CO-8917, Sigma), leupeptin (cat. # L-2023, Sigma) and trans-epoxysuccinyl-1-leucylamido(4-guanidino)butane-E64 (cat.# E-3132, Sigma) at a final concentration of 20 nM. No inhibition of 4HBS activity was observed with any of the three compounds.

Developmental Expression of 4HBS in V. planifolia Organs and Embryo Culture

Protein gel blot analysis indicated that a 28 kDa protein recognized by anti-(4HBS) serum was present in stems, leaves and pods and embryo cultures of V. planifolia, as shown in FIG. 8. Likewise, these tissues all contained 4HBS activity (FIG. 10). In a more detailed developmental study, the levels of 4HBS protein were similar in young mature pods and pods that had been “cured” for up to 12 weeks, but the protein had completely disappeared from pods that had been cured for 24 weeks-and contained large amounts of vanillin aglycone. These data reflected the levels of 4HBS activity in the pods of different stages of development and “curing”.

Because of the sequence similarity of the V. planifolia 4HBS to cysteine proteases, we determined whether the enzyme could be regulated by treatments known to induce or repress cysteine proteases in other plant species. V. planifolia embryo cultures were treated with 10⁻⁵ M, 10⁻⁶ M or 3×10⁻⁸ M gibberellic acid (GA₃), which has been reported to induce cysteine protease expression during germination of rice and barley seeds, and to represses senescence-induced cysteine protease expression in pea ovaries. The cultures were also exposed to 10⁻⁵ M and 10×10⁻⁸ M abscisic acid (ABA), which reduces cysteine protease expression in barley aleurone layers. Twenty four hours after treatment, cultures were harvested and extracts subjected to in-gel protease assay, protein gel blot analysis, and determination of 4HBS activity. The results indicate that treatment of the cultures with GA₃did indeed induce several protease activities, as determined by in-gel protease assay, but that the 28 kDa 4HBS protein was not induced, as shown by parallel protein gel blot assay, and that no protease activity corresponded to a 28 kDa protein. 4HBS transcripts were likewise not induced by GA₃ treatment. Treatment of embryo cultures with ABA had no effect on protease or 4HBS activities.

Cellular Localization of 4HBS in V. planifolia

Immunolocalization of 4HBS was carried out on sections of mature, freshly harvested vanilla beans by using anti-4HBS-M serum. Cross sections revealed that 4HBS-M antigenicity was localized in large vacuolar bodies and cytoplasmic vesicles. Sections treated with preimmune serum were not labeled.

Discussion

The chain-shortening reaction in vanillin biosynthesis proceeds via a non-oxidative pathway. Previous studies on the enzymes leading to 4-hydroxybenzoate derivatives in plants have led to proposals for at least three different mechanisms for the chain-shortening reaction. The present data provide the first example of the molecular characterization of a plant chain-shortening enzyme, and provide confirmation for a non-oxidative mechanism involving a hydrolase activity that proceeds by hydration of the side chain 2,3 double bond of 4-coumaric acid with subsequent cleavage of the side chain to yield acetate and 4-hydroxybenzaldehyde. This reaction, which has been previously proposed as the first step in formation of 4-hydroxybenzoic acid in L. erythrorhizon and carrot cell cultures, is presumed to involve the unstable 4-hydroxyphenyl-β-hydroxypropionic acid (FIG. 1) as an intermediate, although this compound was not detected in the present work.

One study on 4-hydroxybenzoate synthesis in L. erythrorhizon provided convincing evidence for a pathway involving oxidation and cleavage of 4-coumaroyl CoA to 4-hydroxybenzoyl CoA and acetyl CoA in a thioclastic reaction with requirement for NAD. In this study, non-oxidative formation of 4-hydroxybenzaldehyde could also be observed, although at much reduced rates compared to the oxidative conversion. It was concluded that the non-oxidative pathway could represent either an alternative route to 4-hydroxybenzoic acid, or could be an artifact of the assay.

In the present study, no chain shortening of hydroxycinnamoyl CoA derivatives were demonstrated in V. planifolia extracts using the assay conditions previously reported for this type of reaction. The molecular characterization of the V. planifolia 4HBS provides conclusive evidence for the non-oxidative pathway, although the oxidative pathway can occur in plants and, in L. erythrorhizon, it is possible that both pathways may operate.

V. planifolia 4HBS Closely Resembles a cysteine Protease According to its Sequence:

V. planifolia 4HBS possesses many of the characteristics of a monocot cathepsin-like cysteine protease. The complete open reading frame contains an N-terminal signal peptide for entry to the secretory system, a vacuolar sorting signal of identical sequence to that functionally defined as necessary for ligand binding to the plant vacuolar sorting receptor, a preproprotein containing the ERFNIN motif characteristic of cathepsin-like cysteine proteases, and the catalytic triad of conserved cysteine, histidine and asparagines residues found in cysteine proteases. Of the two potential glycosylation sites at positions 122 and 251, the latter is probably not functional because of the exact match between the experimentally determined and predicted molecular weights for the mature protein.

The very high sequence identity between V. planifolia 4HBS and cysteine proteases, particularly those from other monocots, suggests the possibility that 4HBS has evolved from a cysteine protease. Plant EST and genome databases contain large numbers of accessions that are annotated as members of the cysteine, serine, or metalloproteinase families. In most cases there is no functional identification of the encoded protein. It was demonstrated recently that plant acyl transferases involved in the formation of sinapoyl malate in Arabidopsis and acyl glucoses in Lycopersicon pennellii are closely related to serine proteases, providing a precedence for the evolution of natural product pathway enzymes from proteases. Developmental expression and localization of 4HBS The 4HBS activity and immunodetectable protein were detected in leaves, shoots, roots and pods of V. planifolia. Only the beans are known to accumulate significant quantities of vanillin, suggesting that 4HBS expression in the other organs is associated with production of other benzoate acid derivatives.

Vanilla flavor compounds are first synthesized in mature beans, where they accumulate as the corresponding glucosides, presumably in the vacuole, in secretory cells lining the seed space of the bean pod. 4HBS is localized to these cell types. Once the beans are removed from the vine, they undergo a process of senescence, during which the glucosides are hydrolyzed to the aglycones, leading to flavor development. The 4HBS protein and enzymatic activity are maximally expressed in young mature pods, and disappear at the stage of pod senescence associated with accumulation of free vanillin.

### Methods

Plant materials

Vanilla planifolia Plant Material:

Freshly harvested vanilla stem, leaves, roots, beans were frozen immediately in liquid nitrogen and stored at −80° C.

Embryo Cultures:

To initiate the cultures, green V. planifolia beans (4-5 month old) were sterilized, placed in petri dishes, and transferred to fresh semi-solid media every two weeks. The seeds embedded in the beans germinate after 3 to 6 months. The embryo cultures, which contain differentiated cell aggregates, were then grown in petri dishes or propagated shake flasks.

Cultures were maintained in media containing Gamborg's (Gamborg et al., 1968) B-5 salts, 20 g/L sucrose, vitamins, 42 mg/L Cefotaxime sodium, and 33 mg/L Vancomycin HCl; semi-solid media also contained 0.8% agar. The vitamin formulation was: L-glycine 2.0 mg/L, Myoinositol 50 mg/L, thiamine HCl 2.0 mg/L, nicotinic acid 0.5 mg/L, D-biotin 0.25 mg/L, pyroxidine HCl 0.25 mg/L, boric acid 1.5 mg/L, zinc sulfate 1.5 mg/L, cupric sulfate 0.05 mg/L. Cultures were grown in light (at 50 mE sec⁻¹ m⁻²) at 25° C. on a gyrotory shaker at 180 RPM.

Cultures grown in liquid media were subcultured at approximately two week intervals by sieving the cultures, cutting each aggregate into several pieces, and transferring 4-5 g of fresh cells to every 100 ml of new media. Cultures grown in petri dishes were subcultured every four to six weeks (Havkin-Frenkel et al 1996). All chemicals were from Sigma.

Crude Extract Preparation and Assay of 4HBS were Performed as Described in Example 12.

Purification of 4HBS

All procedures were carried out at 4° C. The enzyme was extracted from the tissue with 0.1 M HEPES buffer pH 8.0 containing 10 mM DTT. Ammonium sulfate was added to the crude enzyme preparation to 40% saturation, the resulting precipitate was discarded, and the supernatant fractionated by FPLC on Phenyl-Sepharose Cl 6B. The column was equilibrated in 10 mM Tris-HCl pH 7.0 containing 10 mM DTT (buffer A) with 2M ammonium sulfate. Crude extract in 40% saturated ammonium sulfate was applied to the column, which was the washed with buffer A containing 2 M ammonium sulfate until all unbound proteins were eluted. 4HBS activity was eluted in 0.8 M ammonium sulphate in buffer A. Fractions containing 4HBS activity were pooled, concentrated and desalted using Amicon membrane concentrators with a cut off range of 10,000 Da.

The desalted 4HBS fraction was applied to a MonoQ FPLC ion exchange column equilibrated with 50 mM Tris-HCl pH 7.5 containing 10 mM DTT (buffer B). The 4HBS activity was eluted using a linear gradient of 0.1M NaCl in buffer B and 1 ml fractions were collected. In a separate protocol in which the 4HBS was run a second time through Mono Q, a 0.25 M NaCl gradient at pH 8.0 was used, and 0.25 ml samples were collected. Fractions containing 4HBS activity were pooled, concentrated and desalted as described above.

The 4HBS fraction from MonoQ was further fractionated on a Superdex HR 200 FPLC column equilibrated with 50 mM NaPi buffer pH 7.0 containing 150 mM NaCl and 5 mM DTT. Fractions of 0.6 ml were collected and analyzed for 4HBS activity. The column was calibrated with a range of protein molecular weight standards. Fractions containing 4HBS activity were concentrated, desalted, and stored at 70° C. in 10 mM Tris-HCl pH 7.0 containing 10 mM DTT.

Fractions containing 4HBS activity from MonoQ and Superdex HR 200 were analyzed on 8%-16% SDS—PAGE gels run in Tris-glycine and stained with either Coomassie blue or silver reagent. The 4HBS activity corresponded to a protein of molecular weight 28 kDa based on comparison of activity profiles with PAGE analysis and calibration of the size exclusion column.

Peptide Sequencing

Internal peptide sequences from the putative 4HBS protein were obtained by tryptic digestion of the 28 kDa band on SDS PAGE gels, HPLC/MS analysis of peptides, and automated Edman degradation (Wistar Protein Microsequencing Facility, The Wistar Institute, Philadelphia, Pa.). 4 peptide sequences were obtained, Peptide 1: GVLPVTR (SEQ ID NO:3), Peptide 2: NSWGTNWGDNGYF (SEQ ID NO:4), Peptide 3: GFNLYK (SEQ ID NO:5), and Peptide 4: QGIVSPVK (SEQ ID NO:6).

In-Gel Protease Assays

A method similar to Solomon et al. (1999) and Tibor Pechan et al. (2000) was used. Vanilla embryos and plant material were harvested and frozen in liquid nitrogen. Extracts were prepared by grinding the tissue in 0.1 M HEPES buffer pH 8.0 containing 10 mM DTT. Cell walls and insoluble matter were removed by centrifugation at 4° C. at 10 000 rpm. Laemmli loading sample buffer (Tris-Glycine SDS Loading Sample Buffer): 63 mM Tris HCl, 10% Glycerol, 2% SDS, 0.0025% Bromphenol blue, pH 6.8 (Novex 98/99) was added to the supernatant, and samples were incubated at 37° C. for 4 min before loading on 12% SDS-polyacrylamide gel containing 0.10% gelatin (Criterion Zymogram Gels ,10% Zymogram Gelatin, cat.# 345-0079, BioRAD)) with Tris-Glycine SDS Running Buffer: 25 mM Tris HCl, 192 mM Glysine, 0.1% SDS, pH 8.3 (Novex 98/99). Renaturation was done by two washes with Renaturing Buffer: 10 mM Tris, pH 7.5, and 0.25% Triton X-100 for 45 min each (Mazal Solomon et al., 1999).

The gel was incubated with Developing Buffer: 0.1 M Phosphate Buffer, 2 mM DTT, 5 mM EDTA, pH 6.0 (Henning Scholze and Egbert Tannich, 1994) at 30° C. overnight, stanmed with Coomassie stain: 40% Methanol, 10% Glacial acetic acid, 0.25% Coomassie brilliant blue (R 250) for 30 min and destained with Destaining solution: 7% glacial acetic acid. Active proteases digested the gelatin and appear as white bands.

After staining the gels were scanned on a [Umax] flat- bed scanner with grayscale mode as a transparency, and then converted to a grayscale mode through PhotoShop 5.5 for minor editing with adjustment of lightness and darkness levels.

Cysteine Protease Activity Assay

Protease activity was determined as described by Robinson, Ch. P. Robinson et al. (1997). The assay relies on the cleavage of the chromogenic reagent, BAPNA. The incubation buffer consisted of 25 μl 100 mM BAPNA in dimethyl sulfoxyde, 10 μl sample, 190 μl phenylmethylsulfonyl fluoride buffer consisting of 0.2 mg/ml DTT, 0.5 mg/ml Na₂EDTA, and phenylmethylsulfonyl fluoride in 100 mM phosphate buffer (pH 6.0). Experimental samples, as well as dilution profile of papain were incubated at 37° C. for 1 hr. The reactions were terminated by the addition of 25 μl of glacial acetic acid, determinated at OD 405 nm to determined the amount of p-nitroaniline released. A standard curve was generated from the papain.

cDNA Library Construction and Screening

Oligonucleotides were designed as following: Primer 1a 5′-GGI GTI CTI CCI GTI ACI CG-3′ (SEQ ID NO:7), Primer 1b 5′-CGI GTI ACI GGI AGI ACI CC-3′ (SEQ ID NO:8), Primer 2a 5′-AA(T, C) TCI TGG GGI ACI AA(T, C) TGG GGI GA(T,C) AA (T,C) GGI TA(T,C) TT(T,C) AA-3′ (SEQ ID NO:9), Primer 2b 5′-(C,T)TT (G,A)AA (G,A)TA ICC (G,A)TT (G,A)TC ICC CCA (G,A)TT IGT ICC CCA IGA (G,A)TT-3′ (SEQ ID NO:10) Primer 3a 5′-GGI TT(T,C) AA(T,C) CTI TA(T,C) AA-3′ (SEQ ID NO:11), Primer 3b 5′-(C,T)TT (G,A)TA IAG (G,A)TT (G,A)AA ICC-3′ (SEQ ID NO:12).

PCR reactions were set up as the following primer combinations: 1a:2b, 1a:3b, 2a:1b, 2a:3b, 3a:1b, 3a:2b. The PCR reaction mixture contained 1 μM of each primer, 2.5 Units of Taq DNA polymerase, 200 μM each dNTP, 50 mM KCl, 10 mM Tris-HCl pH 8.3, 1.5 mM MgCl₂₁ 0.1% Triton X 100 and template DNA in a total volume of 100 μl. Template DNA was prepared by boiling a portion of a vanilla embryo culture cDNA library and added 10 μl to the reaction mix. The DNA was denatured at 93° C. for 4 min and amplified through 35 cycles of 45 seconds of denaturation at 94° C., 1 min annealing at 40° C. and 1 min extension at 72° C. and a final extention of 7 min at 72° C.

The PCR reaction containing the primer combination 1a:2b generated a single band PCR product of 800 bp. Gel-purified PCR products were ligated to pGEM-T vector (Promega, Madison, Wis.) and sequenced using Sequencing kit (Pharmacia, Alameda, Calif.).

Expression of 4HBS in E. coli

The p4HBS cDNA was reamplified by PCR using the 4HBS

| | | |
|---|---|---|
| P1a | (GGAATTCCATATGGCAGCTAAGCTCCTCTTC, | (SEQ ID NO:13)) |
| 4HBS P1b | (CGCGGATCCCTACACAGCCACAATGGG, | (SEQ ID NO:14)) |

and which introduce NdeI and Bam-HI restriction sites. The amplification product was digested with NdeI and Bam-HI, gel purified, and ligated to NdeI/Bam-HI digested expression vector pET15b (Novgen, Madison, Wis.). The recombinant plasmids were used to transform E. coli DH5α competent cells. A single colony bearing the pET15b/4HBS was isolated.

To express 4HBS activity, the pET15b/4HBS plasmid was transformed into E. coli BL21(DE3), BL21(DE3) RP, BL21(DE3) RIL, BL21(DE3) pLys S and BL21(DE3) pLysE (Novagen) competent cells. E. coli harboring pET15b without an insert were used as the negative control. A single colony was used to inoculate 10 ml Lb liquid medium containing 100 μg/ml ampicilin. When the embryo culture reached approximately 0.6 OD₆₀₀ units, isopropyl-thiogalactopyranoside (IPTG) was added to a final concentration of 1.0 mM. After an additional 3 h of culture, protein extracts were prepared from the bacterial cells according to manufacturer's protocol (Novagen) and analyzed for 4HBS activity by HPLC analysis, and protease activity by in-gel protease activity assay.

The pET15b/4HBS-M construct, coding truncated (mature) protein (also referred to herein as the mature form of 4HBS, or mature 4HBS (4HBS-M)) lacking the N-terminal region was designed by PCR-based deletions as follows: the pET15b/4HBS was reamplified by PCR using the 4HBS

| | | |
|---|---|---|
| P2a | (CCCATATGCTTCCTGTAACGAGGGATTGG, | (SEQ ID NO:15)) |
| P1b | (CGCGGATCCCTACACAGCCACAATGGG, | (SEQ ID NO:14)) |

and 4HBS P1b which introduce NdeI and Bam-HI site. The amplification product was gel purified and ligated to pBluescript SK(−) expression vector (Stratagene). Two positive clones were sequenced in both directions. The 4HBS-M was digested with NdeI and Bam-HI, gel purified and ligated to NdeI/Bam-HI digested expression vector pET15b (Novgen, Madison, Wis.). The recombinant plasmids were used to transform E. coli DH5a competent cells. A single colony bearing the pET15b/4HBS was isolated. To express 4HBS-M activity, the pET15b/4HBS-M plasmid was transformed into E. coli BL21(DE3) pLys S (Novagen) competent cells. E. coli harboring pET15b without an insert were used as a negative control.

A single colony was used to inoculate 10 ml LB liquid medium containing 100 μg/ml ampicillin. When the embryo culture reached approximately 0.6 OD6₀₀ units, isopropyl-thiogalactopyranoside (IPTG) was added to a final concentration of 1.0 mM. After an additional 3 h of culture, protein extracts were prepared from the bacterial cells according to the manufacturer's protocol (Novagen) and analyzed for 4HBS activity by HPLC analysis, and protease activity by the in-gel protease activity assay.

Expression of 4HBS in Pischia pastoris

4HBS was cloned into pPIC9 Pichia pastoris expression vector (Invitrogen, cat. # K1710-01) according to the manufacturer's protocol. The pPIC9 vector is used for expression of secreted proteins. A major advantage of expressing heterologous proteins as secreted proteins is that Pichia pastoris secretes very low levels of native proteins.

Before transformation of the pPIC9/4HBS plasmid into Pichia pastoris, two independent pPIC9/4HBS E. coli clones were sequenced for insert verification.

Expression of 4HBS in Arabidopsis thaliana.

The pTA7002/4HBS construct was designed as follows: the pET15b/4HBS was reamplified by PCR using the 4HBS

| | | |
|---|---|---|
| P3a | (CCCCTCGAGATGGCAGCTAAGCTCCTCTTC, | (SEQ ID |
| | | NO:16)) |
| P3b | (CCCCACTAGTCTACACAGCCACAATGGG, | (SEQ ID |
| | | NO:17)) |

and 4HBS which introduce XhoI and SpeI restriction sites. The amplification product was gel purified and ligated to pBluescript SK(−) expression vector (Stratagene). Two positive clones were sequenced in both directions. The 4HBS insert was digested with XhoI and SpeI, gel purified and ligated to XhoI/SpeI-digested expression vector pTA7002 (Novgen, Madison, Wis.). The recombinant plasmids were used to transform E. coli DH5-alpha competent cells. A single colony bearing the pET15b/4HBS was isolated.

To express 4HBS activity into Arabidopsis thaliana the pET15b/4HBS plasmid was transformed into C58C1 A. tumefaciens competent cells. A. tumefaciens containing pTA7002 without an insert was used as the negative control.

Agrobacterium tumefaciens-mediated plant transformation with pTA7002 and pTA7002/4HBS was performed by the vacuum infiltration method (Bechtold et al., 1993).

Antibody Production and Protein Gel Blot Analysis

The overexpressed 4HBS-M protein into E. coli was purified and refolded. Polyclonal 4HBS-M antibodies were produced to the refolded protein by Covance Research Products, Inc., PA., USA.

Crude extract (10 μl) (see above) was separated on 12% SDS-PAGE gels, and electrotransferred onto nitrocellulose membranes (Pure Nitrocellulose Membrane, 0.45 μm, BIO-RAD, cat.# 162-0116). The membranes were incubated in blocking buffer: 8% skim milk in PBST buffer (PBS buffer with 0.05% Tween 20) overnight, then incubated in blocking buffer with 4HBS-M anti-serum for 3 hr (1:20,000 dilution), washed 4×10 min with PBST buffer and incubated in PBST buffer with secondary antibody (rabbit IgG conjugated with HRP) (1:10,000 dilution) for 1 hr. After washing with PBST 4×10 min, the signals were detected with ECL Protein gel blotting detection reagents (ECL™ Western blotting detection reagent, Amersham, cat.# RPN-2016) according to the manufacturer's protocol.

Immunolocalization of 4HBS

Immunolocalization of 4HBS was as described in Vincent et al. (2000) with some modifications. Frozen plant material (Vanilla planifolia bean) was thawed, cut into smaller pieces with a razor blade and immediately fixed in a vial containing 4% formaldehyde in PEM (50 mM PIPES; 10 mM EGTA; 5 mM MgSO4) buffer, pH 6.9 and 5% dimethyl sulfoxide (v/v). The tissues were kept in fixative for 2 h followed by extensive washing in PEM buffer. The tissues were then attached to an aluminum block, coated with a thin film of super glue and 150 μm transverse sections were cut using a Vibratome 1000 (Technical Products International, St Louis, Mo.). Sections were retrieved using a pair of fine forceps and carefully transferred onto glass slides. Sections were digested for 10 min in a cocktail of wall degrading enzymes in PEM buffer (1% cellulase; 0.01% pectolyase, and 0.1% BSA) before blocking with 3% (w/v) BSA in PEM buffer for 1.5 hr. This was followed by a 3 h incubation in primary antibody (4NBS antiserum) diluted 1:500 in 1% (w/v) BSA solution in PEM buffer. After extensive washing in PEM buffer, secondary antibody (goat anti-rabbit IgG conjugated to FITC, Sigma Chemicals) was applied to the sections for 2 h. After several washes PME buffer, sections were mounted in 20% Mowiol 4-88 (Calbiochem, La Jolla, Calif.) containing the anti-fading reagent phenylenediamine (0.1%) in Phosphate buffered saline (PBS), pH 8.5.

Alternatively, fixed plant material was dehydrated in a graded series of ethanol, embedded in LR white resin and 1 μm sections obtained using a rotary microtome. Sections were attached to glass slides coated with poly-L-lysine and processed for microscopy as described above.

### EXAMPLE 14

### Cloning of the 4-Hydroxybenzaldehyde Synthase from Vanilla planifolia into the monocot, Creeping Bentgrass

Expression of Vanilla planifolia 4HBS in Creeping Bentgrass

The 4HBS sequence was amplified by PCR using the following oligos: oligo 1.′

| | |
|---|---|
| 5′ GTATCTGAGCTCAAAAATGGCAGCTAAGCTCCTC 3′ | (SEQ ID |
| | NO:18); |
| 5′ CATAGAGGATCCCTACACAGCCACAATGGGATAA 3′ | (SEQ ID |
| | NO:19). |

oligo These oligos contain the 18 and 22 nucleotides at the 5′ and 3′ ends of the 4HBS sequence, respectively. Oligo 1 also has a SacI restriction site and oligo 2 has a BamHI restriction site to facilitate subsequent cloning. The PCR reaction was separated electrophoretically on 1% agarose gel and the PCR-amplified 4HBS fragment was purified. The purified fragment was digested with SacI and BamHI, followed by ethanol precipitation. The fragment was then ligated into the SacI and BamHI digested monocot expression vector resulting in the cloning of the 4HBS sequence between the maize ubiquitin promoter (Christensen and Quail, 1996) and pea rbcS E9 termination sequence (Tumer et al., 1991). The ligated plasmid was used to transform Escherichia coli ElectroMAX DH10B (Life Technologies) cells by electroporation.

The expression vector plasmid was purified from transformed E. coli colonies and the 4HBS sequence was confirmed. A plasmid that had a perfect 4HBS sequence was selected and used to transform creeping bentgrass (Agrostis palustris Huds.) embryogenic callus using particle bombardment. Callus was prepared for transformation by placing 0.5 g tissue on 5.5 cm filter disks in plates of callus culture medium (Lee et al., 1996) containing 0.6 M mannitol for approximately 16 hours prior to the bombardment (Vain et al., 1993). Bombardment was carried out using the Bio-Rad PDS-1000/He Biolistic Delivery System at 1100 psi. Samples were co-bombarded with the 4HBS expression vector and pAcH1 which contains a modified E. coli hph gene for hygromycin resistance (Bilang et al., 1991) controlled by the rice actin promoter (McElroy et al., 1990). Transformed callus was selected on callus culture medium containing 200 ug ml⁻¹ hygromycin. For regeneration, transformed callus colonies were transferred to callus culture medium without auxin but containing 1 mg l⁻¹6-benzyl adenine, and 200 ug ml⁻¹ hygromycin. Shoots were transferred to Phytatrays (Sigma, St. Louis, Mo.) and when large enough, transplanted to potting mix in the greenhouse.

The hygromycin resistant transformants were analyzed by PCR for the presence of the 4HBS coding sequence (Klimyuk et al., 1993). Positive transformants were further analyzed by immunoblotting for production of the 4HBS protein. For protein blots 0.1 gram of leaf tissue was homogenized into 400 uls of 2X SDS sample buffer (125 mM Tris, pH 6.8, 4.6% SDS, 10% b-mercaptoethanol, 20% glycerol, and 0.002% bromophenol blue) (Laemmli, 1970). Samples (15 ul) were subjected to SDS-PAGE and transferred to Nitropure membranes (Fisher) in 10 mM CAPS, pH 11 and 10% methanol. The blots were incubated with a 1:20,000 dilution of the 4HBS antibody. Detection of antibody binding was by chemiluminescence (Western Lightning Chemiluminescence Reagent Plus Kit, Perkin Elmer Life Science Inc).

### REFERENCES

Christensen, A. H., and P. H. Quail. 1996. Ubiquitin promoter-based vectors for high-level expression of selectable and/or screenable marker genes in monocotyledonous plants. Transgenic Res. 5:2133-2138.

Klimyuk, V. I., B. J. Carroll, C. M. Thomas, and J. D. G. Jones. 1993. Alkali treatment for rapid preparation of plant material for reliable PCR analysis. Plant J. 3:493-494.

Laemmli, U. K. 1970. Cleavage of structural proteins during assembly of the head of bacteriophage T4. Nature 227:680-685.

Lee, L., C. L. Laramore, P. R. Day, and N. E. Tumer. 1996. Transformation and regeneration of creeping bentgrass (Agrostis palustris Huds.) protoplasts. Crop Sci. 36:401-406.

McElroy, D., W. Zhang, J. Cao, and R. Wu. 1990. Isolation of an efficient actin promoter for use in rice transformation. Plant Cell 2:163-171.

Tumer, N. E., W. Kaniewski, L. Haley, L. Gehrke, J. K. Lodge, and P. Sanders. 1991. The second amino acid of alfalfa mosaic virus coat protein is critical for coat protein- mediated protection. Proc. Natl. Acad. Sci. USA 88:2331-2335.

Vain, P., M. D. McMullen, and J. J. Finer. 1993. Osmotic treatment enhances particle bombardment-mediated transient and stable transformation of maize. Plant Cell Rep. 12:84-88.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

We claim:

1. A method for improving production of vanillin in cultured Vanillin planifolia, which comprises:
a) providing a tissue culture of said Vanilla planifolia; and
b) supplementing the culture with a compound selected from the group consisting of malic acid, 3,4-dihydroxybenzaldehyde, citric acid, pyruvic acid, oxaloacetic acid, succinic acid, glycosylated lysozyme, and any combination thereof, in an amount effective to improve the vanillin production as compared with cultures not supplemented with the compound.

2. The method of claim 1, wherein the tissue culture is an embryo culture.

3. The method of claim 1, wherein the culture is supplemented with malic acid at a concentration of between about 0.01% and 5% by weight of the culture medium.

4. The method of claim 3, wherein the culture is subjected to mechanical shear stress for 21 days, followed by addition of the malic acid at a concentration of between about 1% and 3% by weight of the culture medium.

5. The method of claim 1, wherein the culture is supplemented with 3,4-dihyrdoxybenzaldehyde at a concentration of between about 0.1 and 5 mM.

6. The method of claim 1, wherein the culture is supplemented with about 0.01 to about 5% by weight of a compound selected from the group consisting of succinic acid, oxaloacetic acid, citric acid and pyruvic acid.

7. The method of claim 1, wherein the culture is supplemented with about 1 to about 100 μg/ml of a glycosylated lysozyme elicitor.

8. Cultured Vanilla planifolia cells, produced by the method of claim 1.

9. The cultured Vanilla planifolia cells of claim 8, which produce at least twice as much vanillin as equivalent cultured cells not supplemented with the compounds.

10. The cultured Vanilla planifolia cells of claim 8, which produce at least ten times as much vanillin as equivalent cultured cells not supplemented with the compounds.

11. A method for improving production of vanillin in cultured Vanilla planifolia, which comprises:
a) providing an embryo culture of said Vanilla planifolia; and
b) subjecting the culture to a stress condition selected from the group consisting of heat stress and mechanical shear stress, in an amount and for a time effective to improve the vanillin production as compared with cultures not subjected to the stress condition.

12. The method of claim 11, wherein the heat stress comprises maintaining the cultures between about 33 and 37° C. for between three and seven days.

13. The method of claim 11, wherein the mechanical shear stress is imposed by placing the cultures in an impeller-driven incubator, under conditions whereby the shear stress is caused.

14. Cultured Vanilla planifolia cells, produced by the method of claim 11.

15. The cultured Vanilla planifolia cells of claim 14, which produce at least twice as much vanillin as equivalent cultured cells not subjected to the stress.

16. A method for improving vanillin production in Vanilla planifolia, which comprises genetically engineering the Vanilla planifolia to overproduce one or more enzymes associated with one or more steps of vanillin biosynthesis in the Vanilla planifolia, the steps selected from the group consisting of: chain shortening of p-coumaric acid to p-hydroxybenzaldehyde; chain shortening of ferulic acid to vanillin; hydroxylation of p-hydroxybenzyl alcohol to 3,4-dihydroxybenzyl alcohol or aldehyde; and methylation of 3,4-dihydroxybenzaldehyde to vanillin.

17. The method of claim 16, wherein the enzymes are selected from the group consisting of: at least one p-hydroxybenzaldehyde synthase; at least one cytochrome p450 monooxygenase; and at least one methyl transferase.

18. The method of claim 16, wherein the enzyme comprises SEQ ID NO:2 or a functional variant thereof.

19. The method of claim 18, wherein the enzyme is encoded by SEQ ID NO:1.

20. The method of claim 16, wherein the genetically engineered Vanilla planifolia is a cell or tissue culture.

21. The method of claim 16, wherein the genetically engineered Vanilla planifolia is a whole plant.

22. A genetically engineered Vanilla planifolia cell produced by the method of claim 16.

23. The cell of claim 22, which produces at least twice as much vanillin as does an equivalent cell which is not comparably genetically engineered.

24. A genetically engineered Vanilla planifolia plant, regenerated from the cell of claim 22.

25. The plant of claim 24, which produces at least twice as much vanillin as does an equivalent plant which is not comparably genetically engineered.

26. A method for improving vanillin accumulation in cell or tissue culture of Vanilla planifolia, which comprises inhibiting production or activity of vanillyl alcohol dehydrogenase in cells comprising the cell or tissue culture, the inhibition resulting in the improved vanillin accumulation.

27. The method of claim 26, wherein the inhibiting comprises genetically engineering the cells to inhibit expression of a gene encoding the vanillyl alcohol dehydrogenase.

28. A genetically engineered Vanilla planifolia cell or tissue culture produced by the method of claim 27.

29. The method of claim 26, wherein the inhibiting comprises treating the culture with an inhibitor of vanillyl dehydrogenase activity.

30. A method for improving vanillin production and accumulation in a Vanilla planifolia cell or tissue culture, which comprises:
a) genetically engineering the Vanilla planifolia to overproduce one or more enzymes associated with one or more steps of vanillin biosynthesis in the Vanilla planifolia, the steps selected from the group consisting of: chain shortening of p-coumaric acid to p-hydroxybenzaldehyde; chain shortening of ferulic acid to vanillin; hydroxylation of p-hydroxybenzyl alcohol to 3,4-dihydroxybenzyl alcohol or aldehyde; and methylation of 3,4-dihydroxybenzaldehyde to vanillin, thereby resulting in the improved vanillin production; and
b) inhibiting production or activity of vanillyl alcohol dehydrogenase in cells of the culture, thereby resulting in the improved vanillin accumulation.

31. A Vanilla planifolia cell or tissue culture produced by the method of claim 30.
